(19) United States
(12) Patent Application Publication
(10) Pub. No.: US 2021/0138080 A1
(43) Pub. Date: May 13, 2021

(54) CONJUGATES

(71) Applicant: GLYKOS BIOMEDICAL OY, Helsinki (FI)

(72) Inventors: Tero SATOMAA, Helsinki (FI); Juhani SAARINEN, Helsinki (FI)

(73) Assignee: GLYKOS BIOMEDICAL OY, Helsinki (FI)

(21) Appl. No.: 17/251,660

(22) PCT Filed: Jun. 19, 2019
(86) PCT No.: PCT/FI2019/050480

§ 371 Date: Dec. 11, 2020

(30) Foreign Application Priority Data
Jun. 29, 2018 (FI) 20185607

(51) Int. Cl. A61K 47/68; A61K 45/06; C07H 19/056; A61P 35/00

(52) U.S. Cl. CPC A61K 47/6807; A61K 47/6855; A61P 35/00; C07H 19/056; A61K 47/6865; A61K 45/06

(57) ABSTRACT

A conjugate is disclosed. The conjugate may comprise a targeting unit for delivery to a target tissue, and a Galectin inhibitor for inhibiting Galectin interaction within the target tissue, wherein the Galectin inhibitor is conjugated to the targeting unit.

### TECHNICAL FIELD

The present disclosure relates to a conjugate.

### BACKGROUND

Immunotherapy for cancer may employ the body's own immune system to recognize and eradicate cancer cells. However, tumour cells, such as cancer cells, may utilize several mechanisms to suppress the activity of cells of the immune system of the subject having the tumour. Means for decreasing the immunosuppressive activity of malignant or cancer cells and/or for boosting immune responses of the subject may therefore improve cancer immunotherapy (Pardoll, Nat. Rev. Cancer 12:252-64, 2012). Combination of targeted therapy to immunotherapy may further improve treatment outcomes (Vanneman & Dranoff, Nat. Rev. Cancer 12:237-51, 2012).

### SUMMARY

A conjugate is disclosed. The conjugate may comprise a targeting unit for delivery to a target tissue, and a Galectin inhibitor for inhibiting Galectin interaction within the target tissue. The Galectin inhibitor may be conjugated to the targeting unit.

### BRIEF DESCRIPTION OF THE DRAWINGS

The accompanying drawings, which are included to provide a further understanding of the embodiments and constitute a part of this specification, illustrate various embodiments. In the drawings:

FIG. 1 illustrates the MALDI-TOF mass spectrum of 6-succinyl-33DFTG reaction products, showing expected mass for both mono-6-succinyl-33DFTG at m/z 771 [M+Na]⁺ and di-6-succinyl-33DFTG at m/z 871 [M+Na]⁺.

FIG. 2 shows the MALDI-TOF mass spectrum of purified di-6-succinyl-33DFTG, with the product ion at m/z 871 [M+Na]⁺.

FIG. 3 shows the MALDI-TOF mass spectrum of di-DBCO-di-6-succinyl-33DFTG, with the product ion at m/z 1387 [M+Na]⁺.

FIG. 4 shows the successful generation of azide-modified trastuzumab, 2 azides/antibody, wherein N-azidoacetylgalactosamine (GalNAz) residues were transferred to N-glycan core N-acetylglucosamine residues with mutant galactosyltransferase reaction after cleaving the N-glycans by endoglycosidase S2. The MALDI-TOF mass spectrum of the heavy chain Fc domain was recorded after isolation of the fragments by Fabricator enzyme digestion, showing the expected m/z values after (A) endoglycosidase digestion and (B) galactosyltransferase reaction. Closed square, GlcNAc; open square with azide, GalNAz; closed triangle, fucose; gray ovals, heavy chain Fc domain fragment.

FIG. 5 shows effective inhibition of Galectin-1 (A and B) and Galectin-3 (C and D) binding to SKOV3 cancer cells by the Galectin inhibitor 33DFTG, as detected with Alexa Fluor 488-conjugated Galectin-1 and Galectin-3 by FACS. Galectin staining drops after incubation with the inhibitor (B and D) compared to untreated cells (A and C). Untreated cells=light grey histogram; Inhibitor-treated cells=dark grey histogram; Control=black line.

FIG. 6 shows effective inhibition of Galectin-1 (A and B) and Galectin-3 (C and D) binding to HSC-2 cancer cells by the Galectin inhibitor 33DFTG, as detected with Alexa Fluor 488-conjugated Galectin-1 and Galectin-3 by FACS. Galectin staining drops after incubation with the inhibitor (B and D) compared to untreated cells (A and C). Untreated cells=light grey histogram; Inhibitor-treated cells=dark grey histogram; Control=black line.

FIG. 7 shows the successful generation of galectin inhibitor-trastuzumab ADCs analyzed by Fabricator digestion of the ADC and MALDI-TOF MS of the isolated antibody fragments as described in Satomaa et al. 2018, Antibodies 7(2):15. Fourth panel (lower panel) shows Fc domain of trastuzumab antibody, wherein the N-glycan was labeled with 1 or 2 azides by reaction with UDP-GAlNAz and Y289L-mutant bovine β1,4-galactosyltransferase 1 (Thermo Fisher Scientific). First panel shows successful conjugation of the Fc domain heavy chain with either 1 or 2 payloads (PL) with structure as shown in Scheme E8-4, DBCO-PEG4-VC-PAB-DMAE-33DFTG. Second panel shows successful conjugation of the Fc domain heavy chain with either 1 or 2 payloads (PL) with structure as shown in Scheme E8-6, DBCO-PEG4-VC-PAB-DMAE-(6-acetyl)33DFTG. Third panel shows successful conjugation of the Fc domain heavy chain with either 1 or 2 payloads (PL) with structure as shown in Scheme E8-5, DBCO-PEG4-VC-PAB-DMAE-(6-succinyl)33DFTG. The generated ADCs comprised forms with DAR=2, DAR=3 and DAR=4.

FIG. 8 shows HIC-HPLC of galectin inhibitor-trastuzumab ADC, performed as in Satomaa et al. 2018. Control ADC DAR=0-8 refers to a mixture of trastuzumab-MC-VC-PAB-MMAE ADCs with drug-to-antibody ratios between 0 and 8. 33DFTG-ADC DAR=4 refers to trastuzumab-DBCO-PEG4-VC-PAB-DMAE-33DFTG DAR=4 ADC, which eluted before 10 ml, between DAR=3 and DAR=4 Control ADCs.

### DETAILED DESCRIPTION

### Outline of Sections

I) Definitions

II) Galectin inhibitors

III) Linker units

IV) Targeting units

V) Stretcher units

VI) Specificity units

VII) Spacer units

VIII) Further linker units

IX) Conjugates

X) Compositions and methods

### I) Definitions

A conjugate is disclosed. The conjugate may comprise

a targeting unit for delivery to a target tissue, and

a Galectin inhibitor for inhibiting Galectin interaction within the target tissue.

Galectins are a class of proteins that are capable of binding specifically to β-galactoside sugars. The structures of the β-galactose binding sites of Galectin-1, 2 and 3 have been described (Lobsanov and Rini, Trends Glycosci Glycotech 1997, 45, 145-154; Seetharaman et al., J Biol Chem 1998, 273, 13047-13052; Saraboji et al., Biochemistry 2012, 51, 296-306). The term “Galectin” may be understood as referring to any S-type lectin, which is a galactoside-recognizing receptor. There are at least 15 Galectins discovered in mammals, encoded by the LGALS genes, of which at least Galectin-1, -2, -3, -4, -7, -8, -9, -10, -12 and -13 have been identified in humans (Essentials of Glycobiology 2017; Chapter 36). Several Galectins have been found or at least implicated to play a role in diseases such as cancer, HIV, autoimmune disease, chronic inflammation, graft vs host disease and allergic reactions. For example, tumours may evade immune responses through Galectin interactions. The roles Galectin interactions may play in e.g. cancer may be quite complex and depend on the specific Galectin.

The Galectin inhibitor may be conjugated to the targeting unit. The Galectin inhibitor may be conjugated to the targeting unit at least partially covalently. For example, it may be conjugated covalently, or partially non-covalently (and partially covalently).

In the context of this specification, the term “target tissue” may refer to any target tissue, for example tumour tissue, to which the conjugate is to be delivered and within which Galectin inhibition is desired.

In an embodiment, the target tissue is a tumour tissue. A tumour tissue may comprise or be at least partially formed of tumour cells.

Many tumours and tumour tissues are known to be formed of not only malignant or cancer cells, but also of non-malignant or non-cancer cells of the subject having the tumour. Such non-malignant or non-cancer cells may be migrated to the tumour tissue, so that they are located within the tumour or the tumour microenvironment or otherwise be intimately associated with the tumour. For example, such non-malignant or non-cancer cells may be located between the malignant or cancer cells, or they may be in direct physical contact with the malignant or cancer cells.

In the context of this specification, the term “tumour cell” may refer to any cell of any cell type that forms a part of or is associated with a tumour or tumour tissue. The term may encompass malignant or cancer cells and, additionally or alternatively, non-cancer or non-malignant cells that form a part of or are associated with the tumour. The term may also encompass any non-cancer or non-malignant cell present in the tumour microenvironment. The tumour cells may include, for example, cells of the immune system. Examples of such tumour cells may include tumour infiltrating immune cells, such as tumour infiltrating lymphocytes, cells of the immune system, cells of the tumour vasculature and lymphatics, as well as fibroblasts, pericytes and adipocytes. Specific examples of such non-cancer tumour cells may include T cells (T lymphocytes); CD8+ cells including cytotoxic CD8+ T cells; CD4+ cells including T helper 1 (TH1) cells, TH2 cells, TH17 cells, Tregs; γδ T lymphocytes; B lymphocytes including B cells and Bregs (B10 cells); NK cells; NKT cells; tumour-associated macrophages (TAMs); myeloid-derived suppressor cells (MDSCs); dendritic cells (DCs); tumour-associated neutrophils (TANs); CD11b+ bone-marrow-derived myeloid cells; fibroblasts including myofibroblasts and cancer-associated fibroblasts; endothelial cells; smooth muscle cells; myoepithelial cells; stem cells including multipotent stem cells, lineage-specific stem cells, progenitor cells, pluripotent stem cells, cancer stem cells (cancer-initiating cells), mesenchymal stem cells and hematopoietic stem cells; adipocytes; vascular endothelial cells; stromal cells; perivascular stromal cells (pericytes); and lymphatic cells including lymphatic endothelial cells (Balkwill et al. 2012. J. Cell Sci. 125:5591-6), provided they form a part of or are associated with the tumour.

In other words, the tumour cells, which thus may form a tumour, may comprise at least malignant or cancer cells and non-cancer or non-malignant cells that form a part of or are associated with the tumour. The target cell may be at least one of the malignant or cancer cells or the non-cancer or non-malignant cells (for example, cells of the immune system). Likewise, the second tumour cell may be at least one of the malignant or cancer cells or the non-cancer or non-malignant cells (for example, cells of the immune system).

The targeting unit may be suitable for delivery to the target tissue, e.g. a tumour tissue, in various ways, for example by being suitable for binding the target tissue, e.g. a cell of the target tissue or a molecule within the target tissue.

In an embodiment, the targeting unit may bind or be capable of binding to a molecule of the target tissue, for example a tumour molecule, thereby facilitating the delivery of the conjugate to the target tissue or to any cells of the target tissue.

In the context of this specification, the term “molecule of the target tissue” may refer to any molecule of any molecule type that forms a part of or is associated (for example, intimately associated) with the target tissue.

In the context of this specification, the term “tumour molecule” may refer to any molecule of any molecule type that forms a part of or is associated (for example, intimately associated) with a tumour or tumour tissue. The term may encompass molecules produced by the malignant or cancer cells and, additionally or alternatively, molecules produced by the non-cancer or non-malignant cells that form a part of or are associated with the tumour or tumour tissue and, additionally or alternatively, molecules that are produced by non-tumour cells and that form a part of or are associated with the tumour or tumour tissue. The term may also encompass any molecule present in the tumour microenvironment. The tumour molecules may include, for example, proteins, lipids, glycans, nucleic acids, or combinations thereof. The tumour molecule may, in some embodiments, be specific to the tumour or tumour tissue or be enriched in the tumour or tumour tissue.

Upon or after delivery to the target tissue and, in some embodiments, binding to a molecule of the target tissue, the conjugate may release the Galectin inhibitor, such that the Galectin inhibitor may, for example, enter or otherwise interact with one or more cells of the target tissue.

In an embodiment, the conjugate is suitable for decreasing, or configured to decrease, Galectin-Galectin ligand interactions.

The conjugate may be suitable for decreasing, or configured to decrease, interactions between tumour cells, for example between cancer or malignant cells, and cells of the immune system.

However, additionally or alternatively, the conjugate may also be suitable for inhibiting, or configured to inhibit, other Galectin functions. For example, a Galectin that has been secreted into an extracellular space of the target tissue may bind to a surface of a cell, for example of a cell of the target tissue. A Galectin inhibitor may thus be suitable for inhibiting, or configured to inhibit, such binding of the secreted Galectin to the surface of the cell.

In an embodiment, the conjugate is a conjugate for decreasing the immunosuppressive activity of cells of the target tissue, for example cells of a tumour tissue.

In the context of this specification, the term “tumour” may refer to a solid tumour, a diffuse tumour, a metastasis, a tumour microenvironment, a group of tumour cells, a single tumour cell or a circulating tumour cell. The term “tumour tissue” may, in the context of this specification, refer to a tissue forming at least a part of a tumour.

In an embodiment, the conjugate is a conjugate for inhibition of inflammation, inhibition of fibrosis, inhibition of angiogenesis, inhibition of infection, inhibition of HIV-1 infection, or inhibition of autoimmune disease or autoimmune reactions in the target tissue.

In an embodiment, the conjugate is a conjugate for inhibition of any Galectin-mediated condition in the target tissue.

In the context of this specification, the term “Galectin inhibitor” may refer to a molecule capable of specifically binding one or more Galectins. The Galectin inhibitor may thereby be capable of inhibiting the function of the Galectin to which it binds or interactions of the Galectin, to which it binds, with one or more other molecules. The Galectin inhibitor may directly bind to and/or interact with a Galectin, for example by attaching, i.e. directly binding, to a Galectin. The Galectin inhibitor may directly bind to and/or interact with the Galectin by non-covalent interactions, such as hydrogen bonds, hydrophobic interactions and/or ionic bonds. In an embodiment, the Galectin inhibitor may be capable of specifically binding to a S-galactose binding site or a Galectin. The Galectin inhibitor may, in an embodiment, be capable of reversibly binding to and thereby inhibiting the Galectin. The Galectin inhibitor may, in an embodiment, be capable of non-covalently binding to and thereby inhibiting a Galectin. Alternatively or additionally, the Galectin inhibitor may be capable of binding irreversibly and/or covalently to a Galectin, thereby inhibiting the Galectin.

In an embodiment, the Galectin inhibitor is not capable of inhibiting glycosylation (at least not to a significant extent).

Examples of Galectins are Galectin-1, Galectin-2, Galectin-3, Galectin-4, Galectin-5, Galectin-6, Galectin-7, Galectin-8, Galectin-9, Galectin-10, Galectin-11, Galectin-12, Galectin-13, Galectin-14, and Galectin-15. The Galectin inhibitor may be capable of specifically binding to and inhibiting one or more of these Galectins.

In an embodiment, the Galectin inhibitor is a Galectin-3 inhibitor. Galectin-3 may be expressed at high levels in various cancers and may thus be considered to be a tumour marker. Galectin-3 may be associated with immunosuppression and thus its inhibition may decrease immunosuppression.

In an embodiment, the Galectin inhibitor is a Galectin-1 inhibitor. Galectin-1 may be expressed at high levels in various cancers and may thus be considered to be a tumour marker. Galectin-1 is associated with immunosuppression and thus its inhibition may decrease immunosuppression.

In an embodiment, the Galectin inhibitor is a Galectin-9 inhibitor. Galectin-9 may be expressed at high levels in various cancers and may thus be considered to be a tumour marker. Galectin-9 is associated with immunosuppression and thus its inhibition may decrease immunosuppression.

In an embodiment, the Galectin inhibitor has an ability to inhibit a plurality of Galectins. In an embodiment, the Galectin inhibitor inhibits a plurality of Galectins including at least Galectin-1 and Galectin-3. In an embodiment, the Galectin inhibitor inhibits a plurality of Galectins including at least Galectin-1 and Galectin-9. In an embodiment, the Galectin inhibitor inhibits a plurality of Galectins including at least Galectin-3 and Galectin-9. In an embodiment, the Galectin inhibitor inhibits a plurality of Galectins including at least Galectin-3 and Galectin-9. In an embodiment, the Galectin inhibitor inhibits a plurality of Galectins including at least Galectin-1, Galectin-3 and Galectin-9.

In this context, the term “a plurality of Galectins” may refer to at least two, i.e. two or more, Galectins; or in some embodiments, at least three Galectins.

In an embodiment, the Galectin inhibitor has an ability to specifically inhibit a Galectin or a group of Galectins; in other words it has substantially higher affinity to the Galectin or the group of Galectins than to other Galectins.

In an embodiment, the Galectin inhibitor is a specific inhibitor of Galectin-1. In an embodiment, the Galectin inhibitor is a specific inhibitor of Galectin-3. In an embodiment, the Galectin inhibitor is a specific inhibitor of Galectin-9. In an embodiment, the Galectin inhibitor is a specific inhibitor of Galectin-1 and Galectin-3. In an embodiment, the Galectin inhibitor is a specific inhibitor of Galectin-1 and Galectin-9. In an embodiment, the Galectin inhibitor is a specific inhibitor of Galectin-3 and Galectin-9. In an embodiment, the Galectin inhibitor is a specific inhibitor of Galectin-1, Galectin-3 and Galectin-9. In the context of this specification, the term “substantially higher affinity” means that there is large difference in the dissociation constantS (Kd) between the two affinities in question. In an embodiment, the difference between the Kd values is at least 5-fold. In an embodiment, the difference between the Kd values is at least 10-fold, at least 100-fold, at least 1000-fold, at least 10000-fold, at least 100000-fold, or at least 1000000-fold.

In the context of this specification, the term “targeting unit” may refer to a group, moiety or molecule capable of recognizing and optionally binding to the target tissue or to a target molecule, for example to a cell of or within the target tissue.

As the Galectin inhibitor and the targeting unit are conjugated at least partially covalently, it may assist in delivering the Galectin inhibitor to the target tissue. The conjugate may also exhibit improved pharmacodynamics and/or pharmacokinetics. Preparing of the conjugate may also be relatively feasible and cost-effective. The conjugate may cause fewer side effects in vivo than e.g. the Galectin inhibitor administered in a non-conjugated or systemic form.

In the context of this specification, the term “to conjugate” or “conjugated” may be understood as referring to linking groups, moieties or molecules to each other at least partially covalently, however such that the linking may, in some embodiments, be arranged at least partially non-covalently. For example, the targeting unit and the Galectin inhibitor may be conjugated via a linker unit, the ends of which are conjugated covalently to the targeting unit and to the Galectin inhibitor.

However, they may be conjugated such that at least a part of the linker unit may comprise units, groups, moieties or molecules that are linked non-covalently, for example via a non-covalent interaction. An example of such a non-covalent interaction may be biotin-avidin interaction or other non-covalent interaction with a sufficient affinity.

A sufficient affinity for the non-covalent linkage or non-covalent interaction may be e.g. one having a dissociation constant (Kd) in the order of nanomolar Kd, picomolar Kd, femtomolar Kd, attomolar Kd, or smaller. In an embodiment, the affinity is substantially the same as the affinity of biotin-avidin interaction. The affinity may be an affinity with a Kd of about 10⁻¹⁴ mol/l, or to a Kd between 10⁻¹⁵ mol/l and 10⁻¹² mol/l (femtomolar), or a Kd below 10⁻¹⁵ mol/l (attomolar). In an embodiment, the affinity is substantially the same as the affinity of an antibody-antigen interaction, such as an affinity having a Kd of about 10⁻⁹ mol/l, or a Kd of between 10⁻¹² mol/l and 10⁻⁹ mol/l (picomolar), or a Kd of between 10⁻⁹ mol/l and 10⁻⁷ mol/l (nanomolar). In an embodiment, the affinity may be an affinity with a Kd that is below 10⁻⁷ mol/l, below 10⁻⁸ mol/l, below 10⁻⁹ mol/l, below 10⁻¹⁰ mol/l, below 10⁻¹¹ mol/l, below 10⁻¹² mol/l, below 10⁻¹³ mol/l, below 10⁻¹⁴ mol/l, or below 10⁻¹⁵ mol/l.

The conjugate may comprise one or more chemical substituents as described by the variables of the chemical formulas of the present disclosure. A person skilled in the art is able to determine what structures are encompassed in the specific substituents based on their names. In the context of this specification, the term “to substitute” or “substituted” may be understood as referring to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known to a person skilled in the art.

In the context of the present specification, the substituents may further comprise certain chemical structures as described in the following embodiments.

In an embodiment, the term “alkyl” means a monovalent moiety obtained or obtainable by removing a hydrogen atom from a carbon atom of a hydrocarbon compound, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, and the like. In an embodiment, the term “C₁₋₁₂ alkyl” means an alkyl moiety having from 1 to 12 carbon atoms.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include isopropyl (C₃), iso-butyl (C₄), sec-butyl (C₄), tert-butyl (C₄), isopentyl (C₅), and neo-pentyl (C₅).

In an embodiment, the term “alkenyl” means an alkyl group having one or more carbon-carbon double bonds. In an embodiment, the term “C₂₋₁₂ alkenyl” means an alkenyl moiety having from 2 to 12 carbon atoms.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

In an embodiment, the term “alkynyl” means an alkyl group having one or more carbon-carbon triple bonds. In an embodiment, the term “C₂₋₁₂ alkynyl” means an alkynyl moiety having from 2 to 12 carbon atoms.

Examples of unsaturated alkynyl groups include, but are not limited to, ethynyl (ethinyl, —C═CH) and 2-propynyl (propargyl, —CH₂—C═CH).

In an embodiment, the term “cycloalkyl” means an alkyl group which is also a cyclyl group; that is, a monovalent moiety obtained by removing a hydrogen atom from an alicyclic ring atom of a cyclic hydrocarbon (carbocyclic) compound. In an embodiment, the term “C₃₋₂₀ cycloalkyl” means a cycloalkyl moiety having from 3 to 20 carbon atoms, including from 3 to 8 ring atoms.

Examples of cycloalkyl groups include, but are not limited to, those derived from:

saturated monocyclic hydrocarbon compounds: cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);

unsaturated monocyclic hydrocarbon compounds: cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and

saturated polycyclic hydrocarbon compounds: norcarane (C₇), norpinane (C₇), norbornane (C₇).

In an embodiment, the term “heterocyclyl” means a monovalent moiety obtained by removing a hydrogen atom from a ring atom of a heterocyclic compound, which moiety has from 3 to 20 ring atoms, of which from 1 to 10 are ring heteroatoms. In an embodiment, each ring has from 3 to 8 ring atoms, of which from 1 to 4 are ring heteroatoms.

In this context, the prefixes (e.g. C₃₋₂₀, C₃₋₈, C₅₋₆, etc.) denote the number of ring atoms, or range of number of ring atoms, whether carbon atoms or heteroatoms. For example, the term “C₅₋₆ heterocyclyl”, means a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted monocyclic heterocyclyl groups include those derived from saccharides, in cyclic form, for example, furanoses (C₅), such as arabinofuranose, ribofuranose, and xylofuranose, and pyranoses (C₆), such as fucopyranose, glucopyranose, mannopyranose, idopyranose, and galactopyranose.

In an embodiment, the term “aryl” means a monovalent moiety obtained by removing a hydrogen atom from an aromatic ring atom of an aromatic compound, which moiety has from 3 to 20 ring atoms. For example, each ring may have from 5 to 8 ring atoms.

In this context, the prefixes (e.g. C₃₋₂₀, C₅₋₈, etc.) denote the number of ring atoms, or range of number of ring atoms, whether carbon atoms or heteroatoms. For example, the term “C₅₋₆ aryl” as used herein, means an aryl group having 5 or 6 ring atoms.

The ring atoms may be all carbon atoms, as in “carboaryl groups”. Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heteroaryls which comprise fused rings, include, but are not limited to:

C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S₁);

C₁₀ (with 2 fused rings) derived from chromene (O₁), isochromene (O₁), chroman (O₁), isochroman (O₁), benzodioxan (O₂) quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₁ (with 2 fused rings) derived from benzodiazepine (N₂);

C₁₃ (with 3 fused rings) derived from carbazole (N₁), dibenzofuran (O₁), dibenzothiophene (S₁), carboline (N₂), perimidine (N₂), pyridoindole (N₂); and,

C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₂S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below. Further, the substituents listed below may themselves be substituents.

Halo: —F, —Cl, —Br, and —I.

Hydroxy: —OH.

Ether: —OR, wherein R is an ether substituent, for example, a C₁₋₁₀ alkyl group (also referred to as a C₁₋₁₀ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₁₀ alkyl group.

Alkoxy: —OR′, wherein R′ is an alkyl group, for example, a C₁₋₁₀ alkyl group. Examples of C₁₋₁₀ alkoxy groups include, but are not limited to, —OMe (methoxy), -OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR′₁)(OR′₂), wherein R′₁ and R′₂ are independently acetal substituents, for example, a C₁₋₁₀ alkyl group, a C₃₋₂₀ heterocyclyl group, or a C₅₋₂₀ aryl group, preferably a C₁₋₁₀ alkyl group, or, in the case of a “cyclic” acetal group, R′₁ and R′₂, taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR′₁), wherein R′₁ is a hemiacetal substituent, for example, a C₁₋₁₀ alkyl group, a C₃₋₂₀ heterocyclyl group, or a C₅₋₂₀ aryl group, preferably a C₁₋₁₀ alkyl group. Examples of hemiacetal groups include, but are not limited to, —CH(OH)(OMe) and —CH(OH)(OEt).

Ketal: —CR′(OR′₁)(OR′₂), where R′₁ and R′₂ are as defined for acetals, and R′ is a ketal substituent other than hydrogen, for example, a C₁₋₁₀ alkyl group, a C₃₋₂₀ heterocyclyl group, or a C₅₋₂₀ aryl group, preferably a C₁₋₁₀ alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe) (OEt).

Hemiketal: —CR′(OH)(OR′₁), where R′₁ is as defined for hemiacetals, and R′ is a hemiketal substituent other than hydrogen, for example, a C₁₋₁₀ alkyl group, a C₃₋₂₀ heterocyclyl group, or a C₅₋₂₀ aryl group, preferably a C₁₋₁₀ alkyl group. Examples of hemiacetal groups include, but are not limited to, —C(Me)(OH) (OMe), —C(Et)(OH)(OMe), —C(Me)(OH)(OEt), and —C(Et)(OH)(OEt).

Oxo (keto, -one): ═O.

Thione (thioketone): ═S.

Imino (imine): ═NR′, wherein R′ is an imino substituent, for example, hydrogen, a C₁₋₁₀ alkyl group, a C₃₋₂₀ heterocyclyl group, or a C₅₋₂₀ aryl group, preferably a C₁₋₁₀ alkyl group. Examples of imino groups include, but are not limited to, ═NH, ═NMe, ═NEt, and ═NPh.

Formyl (carbaldehyde, carboxaldehyde): —C(═O)H.

Acyl (keto): —C(═O)R′, wherein R′ is an acyl substituent, for example, a C₁₋₁₀ alkyl group (also referred to as C₁₋₁₀ alkylacyl or C₁₋₁₀ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₁₀ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR′, wherein R′ is an ester substituent, for example, a C₁₋₁₀ alkyl group, a C₃₋₂₀ heterocyclyl group, or a C₅₋₂₀ aryl group, preferably a C₁₋₁₀ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R′, wherein R′ is an acyloxy substituent, for example, a C₁₋₁₀ alkyl group, a C₃₋₂₀ heterocyclyl group, or a C₅₋₂₀ aryl group, preferably a C₁₋₁₀ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₁₀ alkyl group, a C₃₋₂₀ heterocyclyl group, or a C₅₋₂₀ aryl group, preferably a C₁₋₁₀ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR′₁R′₂, wherein R′₁ and R′₂ are independently amino substituents, for example, hydrogen, a C₁₋₁₀ alkyl group (also referred to as C₁₋₁₀ alkylamino or di-C₁₋₁₀ alkylamino), a C₃₋₂₀ heterocyclyl group, or a C₅₋₂₀ aryl group, preferably H or a C₁₋₁₀ alkyl group, or, in the case of a “cyclic” amino group, R′₁ and R′₂, taken together with the nitrogen atom to which they are attached, form a heterocyclic ring having from 4 to 8 ring atoms. Amino groups may be primary (—NH₂), secondary (—NHR′₁), or tertiary (—NHR′₁R′₂), and in cationic form, may be quaternary (═NR′₁R′₂R′₃). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR′₁R′₂, wherein R′₁ and R′₂ are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′₁ and R′₂, together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR′₁R′₂, wherein R′₁ and R′₂ are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR′₁C(═O)R′₂, wherein R′₁ is an amide substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group, and R′₂ is an acyl substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of acylamide groups include, but are not limited to, —NHC(═O)CH₃, —NHC(═O)CH₂CH₃, and —NHC(═O)Ph. R′₁ and R′₂ may together form a cyclic structure, as in, for example, succinimidyl, maleimidyl, and phthalimidyl:

Aminocarbonyloxy: —OC(═O)NR′₁R′₂, wherein R′₁ and R′₂ are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R′₁)C(═O)NR′₂R′₃ wherein R′₂ and R′₃ are independently amino substituents, as defined for amino groups, and R′₁ is a ureido substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of ureido groups include, but are not limited to, —NHCONH₂, —NHCONHMe, —NHCONHEt, —NHCONMe₂, —NHCONEt₂, NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom.

Imino: ═NR′, wherein R′ is an imino substituent, for example, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of imino groups include, but are not limited to, ═NH, ═NMe, and ═NEt.

Amidine (amidino): —C(═NR′₁)NR′₂, wherein each R′₁ is an amidine substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of amidine groups include, but are not limited to, —C(═NR′₁)NH₂, —C(═NH)NMe₂, and —C(═NMe)NMe₂.

Nitro: —NO2.

Nitroso: —NO.

Azido: —N3.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR′, wherein R′ is a thioether substituent, for example, a C₁₋₁₀ alkyl group (also referred to as a C₁₋₁₀ alkylthio group), a C₃₋₂₀ heterocyclyl group, or a C₅₋₂₀ aryl group, preferably a C₁₋₁₀ alkyl group. Examples of C₁₋₁₀ alkylthio groups include, but are not limited to, —SCH₃ and —SCH₂CH₃.

Disulfide: —SS—R′, wherein R′ is a disulfide substituent, for example, a C₁₋₁₀ alkyl group, a C₃₋₂₀ heterocyclyl group, or a C₅₋₂₀ aryl group, preferably a C₁₋₁₀ alkyl group (also referred to herein as C₁₋₁₀ alkyl disulfide). Examples of C₁₋₁₀ alkyl disulfide groups include, but are not limited to, —SSCH₃ and —SSCH₂CH₃.

Sulfine (sulfinyl, sulfoxide): —S(═O)R′, wherein R′ is a sulfine substituent, for example, a C₁₋₁₀ alkyl group, a C₃₋₂₀ heterocyclyl group, or a C₅₋₂₀ aryl group, preferably a C₁₋₁₀ alkyl group. Examples of sulfine groups include, but are not limited to, —S(═O)CH₃ and —S(═O)CH₂CH₃.

Sulfone (sulfonyl): —S(═O)₂R′, wherein R′ is a sulfone substituent, for example, a C₁₋₁₀ alkyl group, a C₃₋₂₀ heterocyclyl group, or a C₅₋₂₀ aryl group, preferably a C₁₋₁₀ alkyl group, including, for example, a fluorinated or perfluorinated C₁₋₁₀ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR′; wherein R′ is a sulfinate substituent, for example, a C₁₋₁₀ alkyl group, a C₃₋₂₀ heterocyclyl group, or a C₅₋₂₀ aryl group, preferably a C₁₋₁₀ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR′, wherein R′ is a sulfonate substituent, for example, a C₁₋₁₀ alkyl group, a C₃-20 heterocyclyl group, or a C₅₋₂₀ aryl group, preferably a C₁₋₁₀ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R′, wherein R is a sulfinyloxy substituent, for example, a C₁₋₁₀ alkyl group, a C₃₋₂₀ heterocyclyl group, or a C₅₋₂₀ aryl group, preferably a C₁₋₁₀ alkyl group. Examples of sulfinyloxy groups include, but are not limited to, —OS(═O)CH₃ and —OS(═O)CH₂CH₃.

Sulfonyloxy: —OS(═O)₂R′, wherein R′ is a sulfonyloxy substituent, for example, a C₁₋₁₀ alkyl group, a C₃-20 heterocyclyl group, or a C₅-20 aryl group, preferably a C₁₋₁₀ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR′; wherein R′ is a sulfate substituent, for example, a C₁₋₁₀ alkyl group, a C₃₋₂₀ heterocyclyl group, or a C₅₋₂₀ aryl group, preferably a C₁₋₁₀ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR′₁R′₂, wherein R′₁ and R′₂ are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR′₁R′₂, wherein R′₁ and R′₂ are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR'S(═O)₂OH, wherein R′ is an amino substituent, as defined for amino groups. Examples of sulfamino groups include, but are not limited to, —NHS(═O)₂OH and —N(CH₃)S(═O)₂OH.

Sulfonamino: —NR′₁S(═O)₂R′₂, wherein R′₁ is an amino substituent, as defined for amino groups, and R′₂ is a sulfonamino substituent, for example, a C₁₋₁₀ alkyl group, a C₃₋₂₀ heterocyclyl group, or a C₅₋₂₀ aryl group, preferably a C₁₋₁₀ alkyl group. Examples of sulfonamino groups include, but are not limited to, —NHS(═O)₂CH₃ and —N(CH₃)S(═O)₂C₆H₅.

Phosphino (phosphine): —P(R′)₂, wherein R′ is a phosphino substituent, for example, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)(R′)₂, wherein R′ is a phosphinyl substituent, for example, a C₁₋₁₀ alkyl group, a C₃₋₂₀ heterocyclyl group, or a C₅₋₂₀ aryl group, preferably a C₁₋₁₀ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR′)₂, where R′ is a phosphonate substituent, for example, hydrogen, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR′)₂, where R′ is a phosphate substituent, for example, hydrogen, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR′)₂, where R′ is a phosphite substituent, for example, hydrogen, a C₁₋₁₀ alkyl group, a C₃₋₂₀ heterocyclyl group, or a C₅-20 aryl group, preferably hydrogen, a C₁₋₁₀ alkyl group, or a C₅-20 aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR′₁)—N(R′₂)₂, where R′₁ and R′₂ are phosphoramidite substituents, for example, hydrogen, a (optionally substituted) C₁₋₁₀ alkyl group, a C₃₋₂₀ heterocyclyl group, or a C₅-20 aryl group, preferably hydrogen, a C₁₋₁₀ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR′₁)—N(R′₂)₂, where R′₁ and R′₂ are phosphoramidate substituents, for example, hydrogen, a (optionally substituted) C₁₋₁₀ alkyl group, a C₃₋₂₀ heterocyclyl group, or a C₅₋₂₀ aryl group, preferably hydrogen, a C₁₋₁₀ alkyl group, or a C₅-20 aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

In an embodiment, the term “alkylene” means a bidentate moiety obtained by removing two hydrogen atoms, either both from the same carbon atom, or one from each of two different carbon atoms, of a hydrocarbon compound, which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH₂CH₂CH₂CH₂-(heptylene).

Examples of branched saturated C₃₋₁₂ alkylene groups include, but are not limited to, —CH(CH₃) CH₂—, —CH(CH₃) CH₂CH₂—, —CH(CH₃) CH₂CH₂CH₂—, —CH₂CH(CH₃) CH₂—, —CH₂CH(CH₃) CH₂CH₂—, —CH(CH₂CH₃)—, —CH(CH₂CH₃) CH₂—, and —CH₂CH(CH₂CH₃) CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃-12 cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

In an embodiment, the term “glycoside” means a carbohydrate or glycan moiety that is joined by a glycosidic bond.

The glycosidic bond may be an O-, N-, C- or S-glycosidic bond, meaning that the bond is formed to the anomeric carbon of the glycan moiety by an oxygen, nitrogen, carbon or sulphur atom, respectively. The glycosidic bond may be an acetal bond. The glycan may be any monosaccharide, disaccharide, oligosaccharide or polysaccharide, and it may be further substituted by any of the substituents listed above.

Examples of glycoside groups include, but are not limited to, β-D-O-galactoside, N-acetyl-β-D-O-galactosaminide, N-acetyl-α-D-O-galactosaminide, N-acetyl-β-D-O-glucosaminide, N-acetyl-β-D-N-glucosaminide, β-D-O-glucuronide, α-L-O-iduronide, α-D-O-galactoside, α-D-O-glucoside, α-D-C-glucoside, β-D-O-glucoside, α-D-O-mannoside, β-D-O-mannoside, β-D-C-mannoside, α-L-O-fucoside, β-D-O-xyloside, N-acetyl-α-D-O-neuraminide, lactoside, maltoside, dextran, and any analogue or modification thereof.

In an embodiment, an anomeric bond of a glycan moiety may be represented by a wavy line, which indicates that the stereochemistry of the anomeric carbon is not defined and it may exist in either the R or S configuration, in other words beta or alpha configuration, meaning that when the glycan is drawn as a ring the bond may be directed either above or below the ring. In a further embodiment, if the anomeric carbon is drawn with a wavy bond to a hydroxyl group (thus forming a hemiacetal) the wavy bond indicates that the glycan can also exist in the open-ring form (aldehyde or ketone).

In an embodiment, the term “polyethylene glycol” means a polymer comprising repeating “PEG” units of the formula [CH₂CH₂O]ₙ. In an embodiment, the term “PEG₁₋₅₀” means polyethylene glycol moiety having from 1 to 50 PEG units. In an embodiment, the term “substituted polyethylene glycol” means a polyethylene glycol substituted with one or more of the substituents listed above. In an embodiment, the term “branched polyethylene glycol” means a polyethylene glycol moiety substituted with one or more of polyethylene glycol substituents forming a branched structure.

The conjugate may be represented by formula I:

[D-L]ₙ-T Formula I

wherein D is the Galectin inhibitor, T is the targeting unit, L is a linker unit linking D to T at least partially covalently, and n is at least 1.

In formula I, when n is greater than 1, each D may, in principle, be selected independently. Each L may likewise be selected independently.

In formula I, n may be an integer, for example an integer of at least 1.

In formula I, n may be in the range of 1 to about 20, or 1 to about 15, or 1 to about 10, or 2 to 10, or 2 to 6, or 2 to 5, or 2 to 4, or 3 to about 20, or 3 to about 15, or 3 to about 10, or 3 to about 9, or 3 to about 8, or 3 to about 7, or 3 to about 6, or 3 to 5, or 3 to 4, or 4 to about 20, or 4 to about 15, or 4 to about 10, or 4 to about 9, or 4 to about 8, or 4 to about 7, or 4 to about 6, or 4 to 5; or about 7-9; or about 8, or 1, 2, 3, 4, 5, 6, 7, 8, 9, 10, 11, 12, 13, 14, 15, 16, 17, 18, 19, or 20; or in the range of 1 to about 1000, or 1 to about 2000, or 1 to about 400, or 1 to about 200, or 1 to about 100; or 100 to about 1000, or 200 to about 1000, or 400 to about 1000, or 600 to about 1000, or 800 to about 1000; 100 to about 800, or 200 to about 600, or 300 to about 500; or 20 to about 200, or 30 to about 150, or 40 to about 120, or 60 to about 100; over 8, over 16, over 20, over 40, over 60, over 80, over 100, over 120, over 150, over 200, over 300, over 400, over 500, over 600, over 800, or over 1000; or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 52, 54, 56, 58, 60, 62, 63, 64, 66, 68, 70, 72, 74, 76, 78, 80, 82, 84, 86, 88, 90, 92, 94, 96, 98, 100, 110, 120, 130, 140, 150, 160, 170, 180, 190, 200, 220, 240, 260, 280, 300, 350, 400, 450, 500, 550, 600, 650, 700, 750, 800, 850, 900, 950, 1000, 1100, 1200, 1300, 1400, 1500, 1600, 1700, 1800, 1900, or 2000, or greater than 2000.

### II) Galectin Inhibitors

Various Galectin inhibitors may be known, examples and embodiments of which are described below. However, other Galectin inhibitors may also be contemplated.

In an embodiment, the Galectin inhibitor is selected from the group of galactose, a 3-substituted galactose, a β-D-galactoside, a galactoside, a 3-substituted galactoside, a β-D-galactoside, a 3-substituted β-D-galactoside, lactose, a 3′-substituted lactose, a lactoside, a 3′-substituted lactoside, N-acetyllactosamine, a 3′-substituted N-acetyllactosamine, an N-acetyllactosaminide, a 3′-substituted N-acetyllactosaminide, N,N′-di-N-acetyllactosediamine, a 3′-substituted N,N′-di-N-acetyllactosediamine, an N,N′-di-N-acetyllactosediaminide, a 3′-substituted N,N′-di-N-acetyllactosediaminide, a taloside, a 3′-substituted taloside, a β-D-taloside, a 3′-substituted β-D-taloside, a mannoside, a 3′-substituted mannoside, a β-D-mannoside, a 3′-substituted β-D-mannoside, thiodigalactose (TDG), a 3-substituted thiodigalactose, a 3,3′-disubstituted thiodigalactose, 3,3′-dideoxy-3,3′-bis-[4-(3-fluorophenyl)-1H-1,2,3-triazol-1-yl]-1,1′-sulfanediyl-di-β-D-galactopyranoside (33DFTG or TD139), 6-acyl-33DFTG, 6-succinyl-33DFTG, di-6-acyl-33DFTG, di-6-succinyl-33DFTG, a 6-substituted 33DFTG, a 6,6′-disubstituted 33DFTG, (E)-methyl-2-phenyl-4-(β-D-galactopyranosyl)-but-2-enoate, Gal1-4Fuc, a 3′-substituted Gal1-4Fuc, GM-CT-01, GR-MD-02, a pectin, reduced pectin, modified citrus pectin, GCS-100, a poly-N-acetyllactosaminide, lactulose, a lactuloside, a 3′-substituted lactulose, a 3′-substituted lactuloside, lactulosyl-L-leucine, a 3′-substituted lactulosyl-L-leucine, a Galectin-binding peptide, a Galectin-binding peptidomimetic, anginex (βpep-25), 6DBF7, DB16, DB21, PTX008 (0118/OTX008), PTX009 (1097a), a Galectin-binding molecule that inhibits Galectin-Galectin ligand interaction, a Galectin-binding antibody, a Galectin-binding antibody fragment, a Galectin-binding nanobody, an RNAi inhibiting Galectin expression, a soluble Galectin, a soluble Galectin fragment, an oxidized Galectin, an oxidized Galectin fragment, GB1107, and any analog, modification, combination or multivalent combination thereof.

In an embodiment, the multivalent combination is a dimer of a galectin inhibitor. In an embodiment, the dimer of a galectin inhibitor is dimer of 33DFTG, dimer of 6-succinyl-33DFTG or dimer of 6-acetyl-33DFTG. In an embodiment, the dimer is conjugated (i.e. the two Galectin inhibitor moieties are conjugated) with a spacer. In an embodiment, the spacer is a polyethylene glycol (PEG) chain.

In an embodiment, the multivalent combination is a trimer of a galectin inhibitor. In an embodiment, the trimer of a galectin inhibitor is trimer of 33DFTG, trimer of 6-succinyl-33DFTG or trimer of 6-acetyl-33DFTG. In an embodiment, the trimer is conjugated with a spacer. In an embodiment, the spacer is a polyethylene glycol (PEG) chain.

In an embodiment, the multivalent combination is a tetramer of a galectin inhibitor. In an embodiment, the tetramer of a galectin inhibitor is tetramer of 33DFTG, tetramer of 6-succinyl-33DFTG or tetramer of 6-acetyl-33DFTG. In an embodiment, the tetramer is conjugated with a spacer. In an embodiment, the spacer is a polyethylene glycol (PEG) chain.

In the context of this specification, the term “3-substituted” or “6-substituted” may mean that the structure has a substituent joined to the atom in the 3-position or 6-position, respectively, of either the central ring of a monosaccharide inhibitor or a monosaccharide analog inhibitor, or the reducing terminal ring (drawn on the right-hand side in molecular structures) of a disaccharide inhibitor or a disaccharide analog inhibitor. In the context of this specification, the term “3′-substituted” or “6′-substituted” may mean that the structure has a substituent joined to the atom in the 3-position or 6-position, respectively, of the non-reducing terminal ring (drawn on the left-hand side in molecular structures) of a disaccharide inhibitor or a disaccharide analog inhibitor. In the context of this specification, the term “3,3′-disubstituted” or “6,6′-disubstituted” means that the structure has a substituent joined to the atom in the 3-position or 6-position, respectively, of the both rings of the disaccharide inhibitor or the disaccharide analog inhibitor.

[chemical structure]

In an embodiment, the Galectin inhibitor is selected from the group of molecules described in Blanchard et al. 2016 (Expert Opinion on Therapeutic Patents 26, issue 5; text, FIG. 1 and Table 1).

In an embodiment, the Galectin inhibitor is selected from the group of molecules described in any of the patent documents US20030109464, U.S. Pat. Nos. 9,050,352, 6,849,607B2, 7,700,763, US20140336146, WO2014067986, U.S. Pat. Nos. 7,012,068, 7,893,252, 8,722,645, 8,658,787, 8,962,824, US20140086932, US20140235571, US20150147338, U.S. Pat. No. 8,877,263, US20150133399, US20030004132, US20040121981, US20060014719, US20060074050, US2007010438, WO2006128027, U.S. Pat. Nos. 7,339,023, 8,716,343, WO2012131079, WO2014070214, EP2858681, WO2012061395, U.S. Pat. No. 9,034,325, WO2015013388, U.S. Pat. Nos. 8,968,740, 7,662,385, 7,964,575, EP2771367, US20070185014, US20100004163, WO2002089831, U.S. Pat. Nos. 5,948,628, 6,225,071, 8,598,323, 6,890,531, 8,513,208, US20040023855, TW201410702A, and WO2018011093.

In an embodiment, the Galectin inhibitor is represented by formula II:

[chemical structure]

wherein W is O, S, NH, NY₁, CH₂, CY₁H or C(Y₁)₂;

X is O, S, S(═O), S(═O)₂, NH, NY₁, CH₂, CY₁H, C(Y₁)₂ or a bond;

R₁ is H, a saccharide, a saccharide substituted with L′, Z, M, a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl, a substituted C₆-C₂₀ aryl or L′;

R₂ is H, OH, OZ, OM, NHCOCH₃, NHZ, NHM or L′;

R₃ is H, OH, OZ, OM, NHZ, NHM, L′ or Y₃;

R₄ is H, OH, OZ, OM or L′;

R₅ is H, CH₂, a saccharide, a C₁-C₁₀ alkyl, a substituted C₁-C₁₀ alkyl, a C₂-C₁₀ alkenyl, a substituted C₂-C₁₀ alkenyl, a C₂—C₁₀ alkynyl, a substituted C₂-C₁₀ alkynyl, a C₆-C₂₀ aryl, a substituted C₆-C₂₀ aryl or a bond;

Y₅ is either absent or H, OH, OZ, OM or L′;

L′ is a bond to L;

M is a removable masking substituent, independently selected from the group of an acetal, hemiacetal, ketal, hemiketal, imino, formyl, acyl, carboxy, thiocarboxy, thiolocarboxy, thionocarboxy, imidic acid, hydroxamic acid, ester, acyloxy, oxycarboyloxy, amino, amido, thioamido, acylamido, aminocarbonyloxy, ureido, guanidino, tetrazolyl, imino, amidine, nitro, nitroso, azide, cyano, isocyano, cyanato, isocyanato, thiocyano, isothiocyano, sulfhydryl, thioether, disulfide, sulfine, sulfone, sulfinic acid, sulfonic acid, sulfinate, sulfonate, sulfinyloxy, sulfonyloxy, sulfate, sulfamyl, sulfonamido, sulfamino, sulfonamino, phospho, phosphinic acid, phosphonate, phosphoric acid, phosphate, phosphorous acid, phosphite, phosphoramidite, or phosphoramidate substituent, or a glycoside or peptide substituent; each Z is independently selected from the group of a C₁-C₁₀ acyl or a substituted C₁-C₁₀ acyl;

each Y₁ is independently selected from a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl and a substituted C₆-C₂₀ aryl;

with the proviso that not more than one of R₁, R₂, R₃, R₄ and Y₅ is L′, and that the Galectin inhibitor D contains not more than one L′; and wherein

Y₃ is a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl and a substituted C₆-C₂₀ aryl, an azide, or a structure described by any one of formulas FY3-A, FY3-B, FY3-C, FY3-D, FY3-E, and FY3-F:

[chemical structure]

wherein the arrow shows the bond to rest of the structure (i.e. the Galectin inhibitor);

[chemical structure]

wherein the arrow shows the bond to rest of the structure; and

wherein R¹, R², R³, R⁴ and R⁵ are independently selected from the group of H, optionally substituted alkyl groups, halogens, optionally substituted alkoxy groups, OH, substituted carbonyl groups, optionally substituted acyloxy groups, and optionally substituted amino groups; wherein two, three, four or five of R¹, R², R³, R⁴ and R⁵ in adjacent positions may be linked to form one or more rings, and the remaining of R¹, R², R³, R⁴ and R⁵ is/are independently selected from the above group;

[chemical structure]

wherein the arrow shows the bond to rest of the structure; and

wherein Y₃a is either O or NH,

Y₃b is selected from the group of CO, SO₂, SO, PO₂, PO, and CH₂, or is a bond, and

Y₃c is selected from the group of:

a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; or

b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamino group, a phenyl group substituted with at least one hydroxy group, a phenyl group substituted with at least one carbonyl group and a phenyl group substituted with at least one substituted carbonyl group, or

c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, or

d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group;

[chemical structure]

wherein the arrow shows the bond to rest of the structure; and

Y_{3d} is selected from the group of CH₂, CO, SO₂, and phenyl or is a bond; Ria is selected from the group of D-galactose, C3-substituted D-galactose, C3-1,2,3-triazol-1-yl-substituted D-galactose, H, a C₁-C₁₀ alkyl, a C₁-C₁₀ alkenyl, a C₆-C₂₀ aryl, an imino group and a substituted imino group; Y₃ₑ is selected from the group of an amino group, a substituted amino group, an alkyl group, a substituted alkyl group, an alkoxy group, a substituted alkoxy group, an alkylamino group, a substituted alkylamino group, a substituted naphthyl group, a thienyl group, and a substituted thienyl group: wherein said substituent is one or more selected from the group consisting of halogen, alkoxy, alkyl, nitro, sulfo, amino, hydroxy or carbonyl group;

[chemical structure]

wherein the arrow shows the bond to rest of the structure; and

Y_{3f} is either CONH or a 1H-1,2,3-triazole ring; and

Y_{3g} is selected from the group of an alkyl group of at least 4 carbons, an alkenyl group of at least 4 carbons, an alkynyl group of at least 4 carbons, a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, a carbamoyl group substituted with an substituted aryl group, a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group, a phenyl group substituted with at least one trifluoromethoxy group, a phenyl group substituted with at least one sulfo group, a phenyl group substituted with at least one hydroxy group, a phenyl group substituted with at least one carbonyl group, a phenyl group substituted with at least one substituted carbonyl group, a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, a naphthyl group substituted with at least one substituted carbonyl group, a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, a heteroaryl group substituted with at least one substituted carbonyl group, a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group;

[chemical structure]

wherein the arrow shows the bond to rest of the structure; and

Y₃ₕ is NH, CH₂, NRₓ or a bond; Y₃ᵢ is CO, SO, SO₂, PO or PO₂H; Y₃ⱼ is selected from the group of an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, an alkyl group of at least 4 carbon atoms Substituted with a halogen, a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxyl group, a phenyl group substituted with a carbonyl group, a phenyl group substituted with a substituted carbonyl group and a phenyl amino group; R_{1b} is H, a saccharide, an alkyl group, an alkenyl group, or an aryl group and wherein Rx is H, an alkyl group, an alkenyl group, an aryl group, a heteroaryl group or a heterocycle.

In an embodiment, the Galectin inhibitor is represented by formula II;

Y₃ is a structure described by formula FY3-G:

[chemical structure]

wherein the arrow shows the bond to rest of the structure (i.e. the Galectin inhibitor);

R₁ is selected from the group of H, a saccharide, a saccharide substituted with L′, Z, M, a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl, a substituted C₆-C₂₀ aryl, L′, 4-methylphenylthio, ethylthio, 3-chlorophenylthio, 4-chlorophenylthio, phenylthio, 3-bromophenylthio, 3-iodophenylthio, 3,4-dichlorophenylthio, 3-chloro-4-cyanophenylthio, 2,3-dichlorophenylthio and 3,4-dichlorophenoxy; and X is a bond to R₁ in either a or B configuration.

In an embodiment, the Galectin inhibitor is represented by formula III:

[chemical structure]

wherein W′ and W″ re each independently selected from the group of O, S, N, NH, NY₁, CH, CH₂, CY₁H and C(Y₁)₂;

R₂′ is H, OH, OZ, OM, NHCOCH₃, NHZ, NHM or L′;

R₃′ is H, OH, OZ, OM, NHCOCH₃, NHZ, NHM, L′ or Y₃′;

R₄′ is either absent or H, OH, OZ, OM and L′;

R₅′ and R₆′ are each independently either absent or selected from the group of H, CH₂, a saccharide, a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl, a substituted C₆-C₂₀ aryl and a bond;

Y₅′ and Y₆′ are each independently either absent or selected from the group of H, OH, OZ, OM and L′;

Y₃′ is a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl and a substituted C₆-C₂₀ aryl, an azide, or a structure described by any one of formulas FY3-A, FY3-B, FY3-C, FY3-D, FY3-E or FY3-F as described above in the context of Formula II;

and wherein the other substituents are as described above in the context of Formula II;

with the proviso that not more than one of R₁, R₂, R₃, R₄, Y₅, R₁′, R₂′, R₃′, R₄′, Y₅′, and Y₆′ is L′, and that the Galectin inhibitor contains not more than one L′.

The wavy bond between C-4 of the second ring and its substituent R′ may point to either above or below the ring. In other words, C-4 may be either in the R or S configuration.

In an embodiment, the Galectin inhibitor is represented by any one of formulas IV to IX:

[chemical structure]

wherein R¹, R², R³, R⁴ and R⁵ are independently selected from the group of H, optionally substituted alkyl groups, halogens, optionally substituted alkoxy groups, OH, substituted carbonyl groups, optionally substituted acyloxy groups, and optionally substituted amino groups; wherein two, three, four or five of R¹, R², R³, R⁴ and R⁵ in adjacent positions may be linked to form one or more rings, and the remaining of R¹, R², R³, R⁴ and R⁵ is/are independently selected from the above group;

[chemical structure]

wherein Y₃a and Y₃a′ are independently either 0 or NH,

Y₃b and Y₃b′ are independently selected from the group of CO, SO₂, SO, PO₂, PO, and CH₂, or is a bond, and

Y₃c and Y₃c′ are independently selected from the group of:

a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; or

b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamino group, a phenyl group substituted with at least one hydroxy group, a phenyl group substituted with at least one carbonyl group and a phenyl group substituted with at least one substituted carbonyl group, or

c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, or

d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group;

[chemical structure]

wherein Y_{3d} is selected from the group of CH₂, CO, SO₂, and phenyl or is a bond; Ria is selected from the group of D-galactose, C3-substituted D-galactose, C3-1,2,3-triazol-1-yl-substituted D-galactose, H, a C₁-C₁₀ alkyl, a C₁-C₁₀ alkenyl, a C₆-C₂₀ aryl, an imino group and a substituted imino group; Y₃ₑ is selected from the group of an amino group, a substituted amino group, an alkyl group, a substituted alkyl group, an alkoxy group, a substituted alkoxy group, an alkylamino group, a substituted alkylamino group, a substituted naphthyl group, a thienyl group, and a substituted thienyl group: wherein said substituent is one or more selected from the group consisting of halogen, alkoxy, alkyl, nitro, sulfo, amino, hydroxy or carbonyl group;

[chemical structure]

Y_{3f} and Y_{3f}′ are each independently either CONH or a 1H-1,2,3-triazole ring; Y_{3g} and Y_{3g}′ are each independently selected from the group of an alkyl group of at least 4 carbons, an alkenyl group of at least 4 carbons, an alkynyl group of at least 4 carbons, a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, a carbamoyl group substituted with an substituted aryl group, a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group, a phenyl group substituted with at least one trifluoromethoxy group, a phenyl group substituted with at least one sulfo group, a phenyl group substituted with at least one hydroxy group, a phenyl group substituted with at least one carbonyl group, a phenyl group substituted with at least one substituted carbonyl group, a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, a naphthyl group substituted with at least one substituted carbonyl group, a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, a heteroaryl group substituted with at least one substituted carbonyl group, a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group;

[chemical structure]

wherein Y₃ₕ is NH, CH₂, NRₓ or a bond; Y₃ᵢ is CO, SO, SO₂, PO or PO₂H; Y₃ⱼ is selected from the group of an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, an alkyl group of at least 4 carbon atoms Substituted with a halogen, a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxyl group, a phenyl group substituted with a carbonyl group, a phenyl group substituted with a substituted carbonyl group and a phenyl amino group; R_{1b} is H, a saccharide, an alkyl group, an alkenyl group, or an aryl group and wherein Rx is H, an alkyl group, an alkenyl group, an aryl group, a heteroaryl group or a heterocycle;

wherein Y₅, X and Y₅′ are as described above.

The wavy bond between C-4 of the second ring and its substituent, or a wavy bond elsewhere in the present specification, means that the stereochemistry is either R or S; in other words the bond may be directed to either above or below the ring.

Examples of Galectin inhibitors represented by the above Formulas are described e.g. in U.S. Pat. No. 9,353,141 (Formula V of the present disclosure); WO 2005/113568 (Formula VI of the present disclosure); WO 2005/113569 (Formula VII of the present disclosure); WO 2010/126435 (Formula VIII of the present disclosure); U.S. Pat. No. 7,230,096 (Formula IX of the present disclosure), which are herein incorporated in their entirety.

The effectiveness or activity and/or selectivity of individual Galectin inhibitors may vary. For example, embodiments in which R₃ and/or R₃′, or corresponding substituents, comprise cyclic and/or hydrophobic groups or moieties, may have a relatively high affinity to one or more Galectins.

In an embodiment, the the Galectin inhibitor is masked with a removable masking substituent (i.e. a removable group), such that the Galectin inhibitor is capable of binding to a Galectin only after removal of the removable masking substituent.

In an embodiment, the Galectin inhibitor D comprises a removable masking substituent M.

Suitable removable masking substituents or groups may include, for example, an ester group, a carbamate group, a glycoside, a hydrazone group, a peptide, a glycoside, or an acetal group.

In an embodiment, when the masking moiety M is comprised in at least one of R₂, R₂′, R₄, R₄′, Y₅ and Y₅′ in any of the Formulas described in this specification, the Kd of the binding of D to a Galectin is sufficiently large so that D is not capable of binding to Galectin, unless M is first removed.

In an embodiment, the Galectin inhibitor D is represented by any one of Galectin inhibitors represented by

Formula II, wherein R₁ is M, or at least one of R₂, R₃, R₄, R₅ or Y₅ is OM or NHM;

Formula III, wherein at least one of R₂′, R₃′, R₄′, Y₅, Y₅′ or Y₆′ is OM or NHM;

Formula IV, Formula V, or Formula VI, wherein at least one of Y₅ or Y₅′ is OM; Formula VII or Formula VIII, or Formula IX, wherein Y₅ is OM.

In an embodiment, the Galectin inhibitor is represented by any one of Formulas II-IX, wherein Y₅ or Y₅′ (where present) is OM.

In an embodiment, the Galectin inhibitor is represented by any one of Formulas II-IX, wherein Y₅ is OM.

In an embodiment, at least one of R₂, R₂′, R₄, R₄′, Y₅ and Y₅′ is OM. In an embodiment, one of R₂, R₂′, R₄, R₄′, Y₅ and Y₅′ is OM. In an embodiment, at least one of R₂ and R₂′ is OM. In an embodiment, one of R₂ and R₂′ is OM. In an embodiment, at least one of R₄ and R₄′ is OM. In an embodiment, one of R₄ and R₄′ is OM. In an embodiment, at least one of Y₅ and Y₅′ is OM. In an embodiment, one of Y₅ and Y₅′ is OM.

In the context of the present specification, the term “capable of binding to Galectin” may mean that the Kd of the binding interaction of the Galectin inhibitor with the Galectin is sufficiently low. A sufficient affinity for being capable of binding to Galectin may be e.g. one having a dissociation constant (Kd) in the order of micromolar Kd, nanomolar Kd, picomolar Kd, or smaller. In an embodiment, the Kd is below 10⁻³ mol/l (about millimolar or smaller). In an embodiment, the Kd is below 10⁻⁴ mol/l, below 10⁻⁵ mol/l, below 10⁻⁶ mol/l, below 10⁻⁷ mol/l, below 10⁻⁸ mol/l, or below 10⁻⁹ mol/l.

Conversely, in an embodiment, when the Galectin inhibitor comprises the removable masking substituent, the Kd may be in the order of milliomolar Kd or larger. In an embodiment, when the Galectin inhibitor comprises the removable group, the Kd is above 10⁻³ mol/l (about millimolar or larger). In an embodiment, the Kd is above 10⁻² mol/l, above 0.1 mol/l, or above 1 mol/l. Embodiments in which the Galectin inhibitor is masked with a removable group, such that the Galectin inhibitor is capable of binding to Galectin only after removal of the removable group, may reduce or avoid binding of the Galectin inhibitor within tissues in which Galectin inhibition is not necessarily desired. The removable group may prevent or reduce interaction of the Galectin inhibitor at off-tumour locations. For example, in a target tissue such as tumour or cancer tissue, the removable group may be cleaved off, after which the Galectin inhibitor may bind to a Galectin within the tumour or cancer tissue. Such embodiments may thus function in a prodrug-like manner.

The removable group may be removable within a cell, for example a cell of the target tissue.

The removable group may be removable by low pH, by reducing conditions, by a protease or a peptidase, or by a glycosidase; for example in a target cell, in a target cell lysosome, in a target cell cytosol, or in a target tissue.

The Galectin inhibitor according to one or more embodiments described in this specification may be conjugated to the targeting unit in various ways.

### III) Linker Units

Various types of linker units may be suitable, and many are known in the art. The linker unit may comprise one or more linker groups or moieties. It may also comprise one or more groups formed by a reaction between two functional groups. A skilled person will realize that various different chemistries may be utilized when preparing the conjugate, and thus a variety of different functional groups may be reacted to form groups comprised by the linker unit L. In an embodiment, the functional groups are selected from the group consisting of sulfhydryl, amino, alkenyl, alkynyl, azidyl, aldehyde, carboxyl, maleimidyl, succinimidyl and hydroxylamino. A skilled person is capable of selecting the functional groups so that they may react in certain conditions.

The terms “linker unit” and “linker” may be used interchangeably in this specification.

The linker unit may be configured to release the Galectin inhibitor after the conjugate, i.e. the targeting unit, is delivered to the target tissue, for example after the targeting unit is bound to the target tissue. The linker unit may, for example, be cleavable. The cleavable linker unit may be cleavable under intracellular conditions, such that the cleavage of the linker unit may release the Galectin inhibitor in the intracellular environment. The cleavable linker unit may be cleavable under conditions of the tumour microenvironment, such that the cleavage of the linker unit may release the Galectin inhibitor in the tumour or tumour tissue.

The linker unit may be non-cleavable.

The linker unit may be cleavable by a cleaving agent that is present in the intracellular environment (e.g., within a lysosome or endosome) or in the tumour microenvironment. The linker unit can be e.g. a peptidyl linker unit that is cleaved by an intracellular peptidase or protease enzyme, for example a lysosomal or endosomal protease, or a peptidase or a protease of the tumour microenvironment. In some embodiments, the peptidyl linker unit is at least two amino acids long or at least three amino acids long. Cleaving agents can include e.g. cathepsins B and D, plasmin, and a matrix metalloproteinase. The peptidyl linker unit cleavable by an intracellular protease or a tumour microenvironment protease may be a Val-Cit linker or a Phe-Lys linker.

The linker unit may be cleavable by a lysosomal hydrolase or a hydrolase of the tumour microenvironment. In an embodiment, the linker unit can comprise a glycosidic bond that is cleavable by an intracellular glycosidase enzyme, for example a lysosomal or endosomal glycosidase, or a glycosidase of the tumour microenvironment. In some embodiments, the glycosidic linker unit comprises a monosaccharide residue or a larger saccharide. Cleaving agents can include e.g. S-glucuronidase, S-galactosidase and R-glucosidase. The glycosidic linker unit cleavable by an intracellular glycosidase or a tumour microenvironment glycosidase may be a β-D-glucuronide linker unit, a S-galactoside linker unit or a S-glucoside linker unit.

The cleavable linker unit may be pH-sensitive, i.e. sensitive to hydrolysis at certain pH values, for example under acidic conditions. For example, an acid-labile linker unit that is hydrolyzable in the lysosome or the tumour microenvironment {e.g., a hydrazone, semicarbazone, thiosemicarbazone, cis-aconitic amide, orthoester, acetal, ketal, or the like) can be used. Such linker units are relatively stable under neutral pH conditions, such as those in the blood, but are unstable at below pH 5.5 or 5.0, or at at below pH 4.5 or 4.0, the approximate pH of the lysosome. In an embodiment, the hydrolyzable linker unit is a thioether linker unit.

The linker unit may be cleavable under reducing conditions, e.g. a disulfide linker unit, examples of which may include disulfide linker units that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT.

The linker unit may be a malonate linker, a maleimidobenzoyl linker, or a 3′-N-amide analog.

The linker unit may be configured to release the Galectin inhibitor outside of the cells of the target tissue.

In an embodiment, the linker unit is configured to release the Galectin inhibitor into an extracellular space of the target tissue after the conjugate is delivered and/or bound to the target tissue.

L, i.e. the linker unit, in Formula I may, in an embodiment, be represented by formula X

—R₇-L₁-Sₚ-L₂-R₈— Formula X

wherein

R₇ is a group covalently bonded to the Galectin inhibitor;

L₁ is spacer unit or absent;

Sₚ is a specificity unit or absent; and

L₂ is a stretcher unit covalently bonded to the targeting unit or absent; and

R₈ is absent or a group covalently bonded to the targeting unit.

R₇ may, for example, be selected from:

—C(═O)NH—,

—C(═O)O—,

—NHC(═O)—,

—OC(═O)—,

—OC(═O)O—,

—NHC(═O)O—, —OC(═O)NH—,

—NHC(═O)NH, and

—O—,

—NH—,

1,2,3-triazole, and

—S—.

The group —O— may in this context be understood as an oxygen atom forming a glycosidic bond between the Galectin inhibitor and L₁, Sₚ, L₂, R₈ or T (whichever present).

R₈ may, for example, be selected from:

—C(═O)NH—,

—C(═O)O—,

—NHC(═O)—,

—OC(═O)—,

—OC(═O)O—,

—NHC(═O)O—,

—OC(═O)NH—,

—NHC(═O)NH,

—NH—,

1,2,3-triazole,

—S—, and

—O—.

The group —O— may also in the context of R₈ be understood as an oxygen atom forming a glycosidic bond between the targeting unit and L₁, L₂ or Sₚ.

### IV) Targeting Units

In an embodiment, the targeting unit is a targeting unit that is capable of binding an immune checkpoint molecule. In an embodiment, the immune checkpoint molecule is any molecule involved in immune checkpoint function. In an embodiment, the immune checkpoint molecule is a checkpoint protein as defined by the NCI Dictionary of Cancer Terms available at https://www.cancer.gov/publications/dictionaries/cancer-terms/def/immune-checkpoint-inhibitor. In an embodiment, the immune checkpoint molecule is a target molecule of an immune checkpoint inhibitor as defined by the NCI Dictionary of Cancer Terms available at https://www.cancer.gov/publications/dictionaries/cancer-terms/def/immune-checkpoint-inhibitor. In an embodiment, the immune checkpoint molecule is any molecule described in Marin-Acevedo et al. 2018, J Hematol Oncol 11:39.

In an embodiment, the immune checkpoint molecule is selected from the group of PD-1, PD-L1, CTLA-4, lymphocyte activation gene-3 (LAG-3, CD223), T cell immunoglobulin-3 (TIM-3), poly-N-acetyllactosamine, T (Thomsen-Friedenreich) antigen, Globo H, Lewis c (type 1 N-acetyllactosamine), Galectin-1, Galectin-2, Galectin-3, Galectin-4, Galectin-5, Galectin-6, Galectin-7, Galectin-8, Galectin-9, Galectin-10, Galectin-11, Galectin-12, Galectin-13, Galectin-14, Galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (Υ_{c}), CD25 (IL-2Rα), and an arginase.

The targeting unit may comprise or be an antibody. For example, the targeting unit may be a tumour cell-targeting antibody, a cancer-targeting antibody and/or an immune cell-targeting antibody. The conjugate may therefore be an antibody-Galectin inhibitor conjugate.

In the context of this specification, the term “antibody” may be understood broadly. For example, an antibody may be e.g. an scFv, a single domain antibody, an Fv, a VHH antibody, a diabody, a tandem diabody, a Fab, a Fab′, a F(ab′)₂, a Db, a dAb-Fc, a taFv, a scDb, a dAb₂, a DVD-Ig, a Bs(scFv)₄-IgG, a taFv-Fc, a scFv-Fc-scFv, a Db-Fc, a scDb-Fc, a scDb-C_{H}3, or a dAb-Fc-dAb. Furthermore, an antibody may be present in monovalent monospecific, multivalent monospecific, bivalent monospecific, or multivalent multispecific forms.

In an embodiment, the targeting unit is a bispecific targeting molecule capable of binding to two different target molecules at the same time. In an embodiment, the bispecific targeting unit is a bispecific antibody.

The targeting unit may, alternatively or additionally, comprise or be a peptide, an aptamer, or a glycan.

The targeting unit may, alternatively or additionally, comprise or be a cancer-targeting molecule, such as a ligand of a cancer-associated receptor. Examples of such cancer-targeting molecules include but are not limited to folate.

The targeting unit may further comprise one or more modifications, such as one or more glycosylations or glycans. For example, antibodies typically have one or more glycans. These glycans may be naturally occurring or modified. The Galectin inhibitor may, in some embodiments, be conjugated to a glycan of the targeting unit, such as an antibody. In some embodiments, the targeting unit may comprise one or more further groups or moieties, for example a functional group or moiety (e.g. a fluorescent or otherwise detectable label).

The targeting unit may comprise or be, for example, a cancer-targeting antibody selected from the group of bevacizumab, tositumomab, etanercept, trastuzumab, adalimumab, alemtuzumab, gemtuzumab ozogamicin, efalizumab, rituximab, infliximab, abciximab, basiliximab, palivizumab, omalizumab, daclizumab, cetuximab, panitumumab, epratuzumab, 2G12, lintuzumab, nimotuzumab and ibritumomab tiuxetan.

The targeting unit may, in an embodiment, comprise or be an antibody selected from the group of an anti-EGFR1 antibody, an epidermal growth factor receptor 2 (HER2/neu) antibody, an anti-CD22 antibody, an anti-CD30 antibody, an anti-CD33 antibody, an anti-Lewis y antibody, an anti-CD20 antibody, an anti-CD3 antibody, an anti-PSMA antibody, an anti-TROP2 antibody and an anti-AXL antibody.

The target molecule may, in an embodiment, comprise or be a molecule selected from the group of EGFR1, epidermal growth factor receptor 2 (HER2/neu), CD22, CD30, CD33, Lewis y, CD20, CD3, PSMA, trophoblast cell-surface antigen 2 (TROP2) and tyrosine-protein kinase receptor UFO (AXL).

The targeting unit may, in an embodiment, comprise or be an immune checkpoint molecule-targeting antibody selected from the group of nivolumab, pembrolizumab, ipilimumab, atezolizumab, avelumab, durvalumab, BMS-986016, LAG525, MBG453, OMP-31M32, JNJ-61610588, enoblituzumab (MGA271), MGD009, 8H9, MED19447, M7824, metelimumab, fresolimumab, IMC-TR1 (LY3022859), lerdelimumab (CAT-152), LY2382770, lirilumab, IPH4102, 9B12, MOXR 0916, PF-04518600 (PF-8600), MED16383, MED10562, MED16469, INCAGN01949, GSK3174998, TRX-518, BMS-986156, AMG 228, MEDI1873, MK-4166, INCAGN01876, GWN323, JTX-2011, GSK3359609, MEDI-570, utomilumab (PF-05082566), urelumab, ARGX-110, BMS-936561 (MDX-1203), varlilumab, CP-870893, APX005M, ADC-1013, lucatumumab, Chi Lob 7/4, dacetuzumab, SEA-CD40, R07009789, and MED19197.

The targeting unit may comprise or be a molecule selected from the group of an immune checkpoint inhibitor, an anti-immune checkpoint molecule, anti-PD-1, anti-PD-L1 antibody, anti-CTLA-4 antibody, or an antibody targeting an immune checkpoint molecule selected from the group of: lymphocyte activation gene-3 (LAG-3, CD223), T cell immunoglobulin-3 (TIM-3), poly-N-acetyllactosamine, T (Thomsen-Friedenreich antigen), Globo H, Lewis c (type 1 N-acetyllactosamine), Galectin-1, Galectin-2, Galectin-3, Galectin-4, Galectin-5, Galectin-6, Galectin-7, Galectin-8, Galectin-9, Galectin-10, Galectin-11, Galectin-12, Galectin-13, Galectin-14, Galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD12 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (Υ_{c}), CD25 (IL-2Rα), and arginase.

The target molecule may comprise or be a molecule selected from the group of an immune checkpoint molecule, PD-1, PD-L1, CTLA-4, lymphocyte activation gene-3 (LAG-3, CD223), T cell immunoglobulin-3 (TIM-3), poly-N-acetyllactosamine, T (Thomsen-Friedenreich antigen), Globo H, Lewis c (type 1 N-acetyllactosamine), Galectin-1, Galectin-2, Galectin-3, Galectin-4, Galectin-5, Galectin-6, Galectin-7, Galectin-8, Galectin-9, Galectin-10, Galectin-11, Galectin-12, Galectin-13, Galectin-14, Galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (Υ_{c}), CD25 (IL-2Rα), and arginase.

### V) Stretcher Units

The term “stretcher unit” may refer to any group, moiety or linker portion capable of linking R₇, L₁, or Sₚ (whichever present) to R₈ (if present) or to the targeting unit. Various types of stretcher units may be suitable, and many are known in the art.

The stretcher unit L₂ may have a functional group that can form a bond with a functional group of the targeting unit. The stretcher unit may also have a functional group that can form a bond with a functional group of either R₇, L1, or Sₚ. Useful functional groups that can be present on the targeting unit, either naturally or via chemical manipulation, include, but are not limited to, sulfhydryl (—SH), amino, hydroxyl, carboxy, the anomeric hydroxyl group of a carbohydrate, and carboxyl. The functional groups of the targeting unit may, in an embodiment, be sulfhydryl and amino. The stretcher unit can comprise for example, a maleimide group, an aldehyde, a ketone, a carbonyl, or a haloacetamide for attachment to the targeting unit.

In one example, sulfhydryl groups can be generated by reduction of the intramolecular disulfide bonds of a targeting unit, such as an antibody. In another embodiment, sulfhydryl groups can be generated by reaction of an amino group of a lysine moiety of an antibody or other targeting unit with 2-iminothiolane (Traut's reagent) or other sulfhydryl generating reagents. In certain embodiments, the targeting unit is a recombinant antibody and is engineered to carry one or more lysines. In certain other embodiments, the recombinant antibody is engineered to carry additional sulfhydryl groups, e.g. additional cysteines.

In an embodiment, the stretcher unit has an electrophilic group that is reactive to a nucleophilic group present on the targeting unit (e.g. an antibody). Useful nucleophilic groups on the targeting unit include but are not limited to, sulfhydryl, hydroxyl and amino groups. The heteroatom of the nucleophilic group of the targeting unit is reactive to an electrophilic group on a stretcher unit and forms a covalent bond to the stretcher unit. Useful electrophilic groups include, but are not limited to, maleimide and haloacetamide groups. For an antibody as the targeting unit, the electrophilic group may provide a convenient site for antibody attachment for those antibodies having an accessible nucleophilic group.

In another embodiment, the stretcher unit has a reactive site which has a nucleophilic group that is reactive to an electrophilic group present on a targeting unit (e.g. an antibody). Useful electrophilic groups on a targeting unit include, but are not limited to, aldehyde and ketone and carbonyl groups. The heteroatom of a nucleophilic group of the stretcher unit can react with an electrophilic group on the targeting unit and form a covalent bond to the targeting unit, e.g. an antibody. Useful nucleophilic groups on the stretcher unit include, but are not limited to, hydrazide, hydroxylamine, amino, hydrazine, thiosemicarbazone, hydrazine carboxylate, and arylhydrazide. For an antibody as the targeting unit, the electrophilic group on the antibody may provide a convenient site for attachment to a nucleophilic stretcher unit.

In an embodiment, the stretcher unit has a reactive site which has an electrophilic group that is reactive with a nucleophilic group present on a targeting unit, such as an antibody. The electrophilic group provides a convenient site for the targeting unit (e.g., antibody) attachment. Useful nucleophilic groups on an antibody include but are not limited to, sulfhydryl, hydroxyl and amino groups. The heteroatom of the nucleophilic group of an antibody is reactive to an electrophilic group on the stretcher unit and forms a covalent bond to the stretcher unit. Useful electrophilic groups include, but are not limited to, maleimide and haloacetamide groups and NHS esters.

In another embodiment, a stretcher unit has a reactive site which has a nucleophilic group that is reactive with an electrophilic group present on the targeting unit. The electrophilic group on the targeting unit (e.g. antibody) provides a convenient site for attachment to the stretcher unit. Useful electrophilic groups on an antibody include, but are not limited to, aldehyde and ketone carbonyl groups. The heteroatom of a nucleophilic group of the stretcher unit can react with an electrophilic group on an antibody and form a covalent bond to the antibody. Useful nucleophilic groups on the stretcher unit include, but are not limited to, hydrazide, oxime, amino, hydrazine, thiosemicarbazone, hydrazine carboxylate, and arylhydrazide.

In some embodiments, the stretcher unit forms a bond with a sulfur atom of the targeting unit via a maleimide group of the stretcher unit. The sulfur atom can be derived from a sulfhydryl group of the targeting unit. Representative stretcher units of this embodiment include those within the square brackets of Formulas Xa and Xb, wherein the wavy line indicates attachment within the conjugate and R¹⁷ is —C₁-C₁₀ alkylene-, —C₁-C₁₀ heteroalkylene-, —C₃-C₈ carbocyclo-, —O—(C₁-C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)—C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)—C₁-C₁₀ alkylene-C(═O)—, —C₃-C₈ heterocyclo-C(═O)—, —C₁-C₁₀ alkylene-(C₃-C₈ heterocyclo)—C(═O)—, —(C₃-C₈ heterocyclo)—C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)—C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—. Any of the R¹⁷ substituents can be substituted or nonsubstituted. In an embodiment, the R¹⁷ substituents are unsubstituted. In another embodiment, the R¹⁷ substituents are optionally substituted. In some embodiments, the R¹⁷ groups are optionally substituted by a basic unit, e.g —(CH₂)ₓNH₂, —(CH₂)ₓNHR^{a}₂, and —(CH₂)ₓNR^{a}₂, wherein x is an integer in the range of 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group.

[chemical structure]

[chemical structure]

In the context of the embodiments of the stretcher unit, the wavy line may (although not necessarily) indicate attachment within the conjugate to either R₇, L₁, or Sₚ, whichever present. The free bond without the wavy line, typically at the opposite end of the stretcher unit, may indicate the bond to the targeting unit.

An illustrative stretcher unit is that of Formula Xa wherein R¹⁷ is —C₂-C₅ alkylene-C(═O)— wherein the alkylene is optionally substituted by a basic unit, e.g —(CH₂)ₓNH₂, —(CH₂)ₓNHR^{a}, and —(CH₂)ₓNR^{a}₂, wherein x is an integer in the range of 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group. Exemplary embodiments are as follows:

[chemical structure]

It will be understood that the substituted succinimide may exist in a hydrolyzed form as shown below:

[chemical structure]

Illustrative stretcher units prior to conjugation to the targeting unit include the following:

[chemical structure]

It will be understood that the amino group of the stretcher unit may be suitably protected by a amino protecting group during synthesis, e.g., an acid labile protecting group (e.g, BOC).

Yet another illustrative stretcher unit is that of Formula Xb wherein R¹⁷ is —(CH₂)₅—:

[chemical structure]

In another embodiment, the stretcher unit is linked to the targeting unit via a disulfide bond between a sulfur atom of the targeting unit and a sulfur atom of the stretcher unit. A representative stretcher unit of this embodiment is depicted within the square brackets of Formula XI, wherein the wavy line indicates attachment within the conjugate and R¹⁷ is as described above for Formula Xa and Xb.

[chemical structure]

In yet another embodiment, the reactive group of the stretcher unit contains a reactive site that can form a bond with a primary or secondary amino group of the targeting unit. Example of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted within the square brackets of Formulas XIIa, XIIb, and XIIc wherein the wavy line indicates attachment within the within the conjugate and R¹⁷ is as described above for Formula Xa and Xb.

[chemical structure]

In yet another embodiment, the reactive group of the stretcher unit contains a reactive site that is reactive to a modified carbohydrate's (—CHO) group that can be present on the targeting unit. For example, a carbohydrate can be mildly oxidized using a reagent such as sodium periodate and the resulting (—CHO) unit of the oxidized carbohydrate can be condensed with a stretcher unit that contains a functionality such as a hydrazide, an oxime, a primary or secondary amine, a hydrazine, a thiosemicarbazone, a hydrazine carboxylate, and an arylhydrazide. Representative stretcher units of this embodiment are depicted within the square brackets of Formulas XIIIa, XIIIb, and XIIIc, wherein the wavy line indicates attachment within the conjugate and R¹⁷ is as described above for Formula Xa and Xb.

[chemical structure]

In some embodiments, it may be desirable to extend the length of the stretcher unit. Accordingly, a stretcher unit can comprise additional components. For example, a stretcher unit can include those within the square brackets of formula XIVa1:

[chemical structure]

wherein the wavy line indicates attachment to the remainder of the conjugate and the free bond to the targeting unit;

and R¹⁷ is as described above. For example, R¹⁷ may be —C₂-C₅ alkylene-C(═O)— wherein the alkylene is optionally substituted by a basic unit, e.g —(CH₂)ₓNH₂, —(CH₂)ₓNHR^{a}, and —(CH₂)ₓNR^{a}₂, wherein x is an integer in the range of 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group; and

R¹³ is —C₁-C₆ alkylene-, —C₃-C₈ carbocyclo-, -arylene-, —C₁-C₁₀ heteroalkylene-, —C₃-C₈ heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)—C₁-C₁₀alkylene-, —C₁-C₁₀alkylene-(C₃-C₈ heterocyclo)-, or —(C₃-C₈ heterocyclo)-C₁-C₁₀ alkylene-. In an embodiment, R¹³ is —C₁-C₆ alkylene-.

The stretcher unit may, in some embodiments, have a mass of no more than about 1000 daltons, no more than about 500 daltons, no more than about 200 daltons, from about 30, 50 or 100 daltons to about 1000 daltons, from about 30, 50 or 100 daltons to about 500 daltons, or from about 30, 50 or 100 daltons to about 200 daltons.

In an embodiment, the stretcher unit forms a bond with a sulfur atom of the targeting unit, for example an antibody. The sulfur atom can be derived from a sulfhydryl group of the antibody. Representative stretcher units of this embodiment are depicted within the square brackets of Formulas XVa and XVb, wherein R¹⁷ is selected from —C₁-C₁₀ alkylene-, —C₁-C₁₀ alkenylene-, —C₁-C₁₀ alkynylene-, carbocyclo-, —O—(C₁-C₈ alkylene)-, O—(C₁-C₈ alkenylene)-, —O—(C₁-C₈ alkynylene)-, -arylene-, —C₁-C₁₀ alkylene-arylene-, —C₂-C₁₀ alkenylene-arylene, —C₂-C₁₀ alkynylene-arylene, -arylene-C₁-C₁₀ alkylene-, -arylene-C₂-C₁₀ alkenylene-, -arylene-C₂-C₁₀ alkynylene-, —C₁-C₁₀ alkylene-(carbocyclo)-, —C₂-C₁₀ alkenylene-(carbocyclo)-, —C₂-C₁₀ alkynylene-(carbocyclo)-, -(carbocyclo)-C₁-C₁₀ alkylene-, -(carbocyclo)-C₂-C₁₀ alkenylene-, -(carbocyclo)-C₂-C₁₀ alkynylene, -heterocyclo-, —C₁-C₁₀ alkylene-(heterocyclo)-, —C₂-C₁₀ alkenylene-(heterocyclo)-, —C₂-C₁₀ alkynylene-(heterocyclo)-, -(heterocyclo)—C₁-C₁₀ alkylene-, -(heterocyclo)—C₂-C₁₀ alkenylene-, -(heterocyclo)-C₁-C₁₀ alkynylene-, —(CH₂CH₂O)ᵣ—, or —(CH₂CH₂O)ᵣ—CH₂—, and r is an integer ranging from 1-10, wherein said alkyl, alkenyl, alkynyl, alkylene, alkenylene, alkynyklene, aryl, carbocycle, carbocyclo, heterocyclo, and arylene radicals, whether alone or as part of another group, are optionally substituted. In some embodiments, said alkyl, alkenyl, alkynyl, alkylene, alkenylene, alkynyklene, aryl, carbocyle, carbocyclo, heterocyclo, and arylene radicals, whether alone or as part of another group, are unsubstituted. In some embodiments, R¹⁷ is selected from —C₁-C₁₀ alkylene-, -carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(carbocyclo)-, -(carbocyclo)-C₁-C₁₀ alkylene-, —C₃-C₈ heterocyclo-, —C₁-C₁₀ alkylene-(heterocyclo)-, -(heterocyclo)—C₁-C₁₀ alkylene-, —(CH₂CH₂)ᵣ—, and —(CH₂CH₂O)ᵣ—CH₂—; and r is an integer ranging from 1-10, wherein said alkylene groups are unsubstituted and the remainder of the groups are optionally substituted.

[chemical structure]

It is to be understood from all the exemplary embodiments that even where not denoted expressly, one or more Galectin inhibitor moieties can be linked to a targeting unit, i.e. n may be 1 or more.

An illustrative stretcher unit is that of Formula XVa wherein R¹⁷ is —(CH₂CH₂O)ᵣ—CH₂—; and r is 2:

[chemical structure]

An illustrative stretcher unit is that of Formula XVa wherein R¹⁷ is arylene- or arylene-C₁-C₁₀ alkylene-. In some embodiments, the aryl group is an unsubstituted phenyl group.

In certain embodiments, the stretcher unit is linked to the targeting unit via a disulfide bond between a sulfur atom of the targeting unit and a sulfur atom of the stretcher unit. A representative stretcher unit of this embodiment is depicted in Formula XVI, wherein R¹⁷ is as defined above.

—S [symbol] S—R¹⁷—C(O) [symbol] Formula XVI

The S moiety in the formula XVI above may refer to a sulfur atom of the targeting unit, unless otherwise indicated by context.

In yet other embodiments, the stretcher unit contains a reactive site that can form a bond with a primary or secondary amino group of the targeting unit, such as an antibody. Examples of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted within the square brackets of Formulas XVIIa and XVIIb, wherein —R¹⁷ is as defined above:

[chemical structure]

In some embodiments, the stretcher unit contains a reactive site that is reactive to a modified carbohydrate's (—CHO) group that can be present on the targeting unit, for example an antibody. For example, a carbohydrate can be mildly oxidized using a reagent such as sodium periodate and the resulting (—CHO) unit of the oxidized carbohydrate can be condensed with a stretcher unit that contains a functionality such as a hydrazide, an oxime, a primary or secondary amine, a hydrazine, a thiosemicarbazone, a hydrazine carboxylate, and an arylhydrazide. Representative stretcher units of this embodiment are depicted within the square brackets of Formulas XVIIIa, XVIIIb, and XVIIIc, wherein —R¹⁷— is as defined as above.

[chemical structure]

In embodiments in which the targeting unit is a glycoprotein, for example an antibody, the glycoprotein, i.e. the targeting unit, may be contacted with a suitable substrate, such as UDP-GalNAz, in the presence of a GalT or a GalT domain catalyst, for example a mutant GalT or GalT domain. Thus the targeting unit may have a GalNAz residue incorporated therein. The Galectin inhibitor may then be conjugated via a reaction with the GalNAz thus incorporated in the targeting unit.

WO/2007/095506, WO/2008/029281 and WO/2008/101024 disclose methods of forming a glycoprotein conjugate wherein the glycoprotein is contacted with UDP-GalNAz in the presence of a GalT mutant, leading to the incorporation of GalNAz at a terminal non-reducing GlcNAc of an antibody carbohydrate. Subsequent copper-catalyzed or copper-free (metal-free) click chemistry with a terminal alkyne or Staudinger ligation may then be used to conjugate a molecule of interest, in this case the Galectin inhibitor, optionally via a suitable linker unit or stretcher unit, to the attached azide moiety.

If no terminal GlcNAc sugars are present on the targeting unit, such as an antibody, endoenzymes Endo H, Endo A, Endo F, Endo D, Endo T, Endo S and/or Endo M and/or a combination thereof, the selection of which depends on the nature of the glycan, may be used to generate a truncated chain which terminates with one N-acetylglucosamine residue attached in an antibody Fc region.

In an embodiment, the endoglycosidase is Endo S, Endo S49, Endo F or a combination thereof.

In an embodiment, the endoglycosidase is Endo S, Endo F or a combination thereof.

Endo S, Endo A, Endo F, Endo M, Endo D and Endo H are known to the person skilled in the art. Endo S49 is described in WO/2013/037824 (Genovis AB). Endo S49 is isolated from Streptococcus pyogenes NZ131 and is a homologue of Endo S. Endo S49 has a specific endoglycosidase activity on native IgG and cleaves a larger variety of Fc glycans than Endo S.

Galactosidases and/or sialidases can be used to trim galactosyl and sialic acid moieties, respectively, before attaching e.g. GalNAz moieties to terminal GlcNAcs. These and other deglycosylation steps, such as defucosylation, may be applied to G2F, G1F, G0F, G2, G1, and G0, and other glycoforms.

Mutant GalTs include but are not limited to bovine beta-1,4-galactosyltransferase I (GalT1) mutants Y289L, Y289N, and Y289I disclosed in Ramakrishnan and Qasba, J. Biol. Chem., 2002, vol. 277, 20833) and GalT1 mutants disclosed in WO/2004/063344 and WO/2005/056783 and their corresponding human mutations.

Mutant GalTs (or their GalT domains) that catalyze the formation of i) a glucose-β(1,4)-N-acetylglucosamine bond, ii) an N-acetylgalactosamine-β(1,4)-N-acetylglucosamine bond, iii) a N-acetylglucosamine-β(1,4)-N-acetylglucosamine bond, iv) a mannose-β(1,4)-N-acetylglucosamine bond are disclosed in WO 2004/063344. The disclosed mutant GalT (domains) may be included within full-length GalT enzymes, or in recombinant molecules containing the catalytic domains, as disclosed in WO2004/063344.

In an embodiment, GalT or GalT domain is for example Y284L, disclosed by Bojarovd et al., Glycobiology 2009, 19, 509.

In an embodiment, GalT or GalT domain is for example R228K, disclosed by Qasba et al., Glycobiology 2002, 12, 691.

In an embodiment, the mutant GalT1 is a bovine β(1,4)-galactosyltransferase 1.

In an embodiment, the bovine GalT1 mutant is selected from the group consisting of Y289L, Y289N, Y289I, Y284L and R228K.

In an embodiment, the mutant bovine GalT1 or GalT domain is Y289L.

In an embodiment, the GalT comprises a mutant GalT catalytic domain from a bovine β(1,4)-galactosyltransferase, selected from the group consisting of GalT Y289F, GalT Y289M, GalT Y289V, GalT Y289G, GalT Y289I and GalT Y289A. These mutants may be provided via site-directed mutagenesis processes, in a similar manner as disclosed in WO 2004/063344, in Qasba et al., Prot. Expr. Pur. 2003, 30, 219 and in Qasba et al., J. Biol. Chem. 2002, 277, 20833 for Y289L, Y289N and Y289I.

Another type of a suitable GalT is α(1,3)-N-galactosyltransferase (α3Gal-T).

In an embodiment, α(1,3)-N-acetylgalactosaminyltransferase is α3GalNAc-T as disclosed in WO2009/025646. Mutation of α3Gal-T can broaden donor specificity of the enzyme, and make it an α3GalNAc-T. Mutation of α3GalNAc-T can broaden donor specificity of the enzyme. Polypeptide fragments and catalytic domains of α(1,3)-N-acetylgalactosaminyltransferases are disclosed in WO/2009/025646.

In an embodiment, the GalT is a wild-type galactosyltransferase.

In an embodiment, the GalT is a wild-type β(1,4)-galactosyltransferase or a wild-type β(1,3)-N-galactosyltransferase.

In an embodiment, GalT is β(1,4)-galactosyltransferase I.

In an embodiment, the β(1,4)-galactosyltransferase is selected from the group consisting of a bovine β(1,4)-Gal-T1, a human β(1,4)-Gal-T1, a human β(1,4)-Gal-T2, a human β(1,4)-Gal-T3, a human β(1,4)-Gal-T4 and β(1,3)-Gal-T5.

In an embodiment, β-(1,4)-N-acetylgalactosaminyltransferase is selected from the mutants disclosed in WO 2016/170186.

The linker unit or the stretcher unit may comprise an alkyne group, for example a cyclic alkyne group, capable of reacting with the azide group of the GalNAz incorporated in the targeting unit, thereby forming a triazole group. Examples of suitable cyclic alkyne groups may include DBCO, OCT, MOFO, DIFO, DIFO2, DIFO3, DIMAC, DIBO, ADIBO, BARAC, BCN, Sondheimer diyne, TMDIBO, S-DIBO, COMBO, PYRROC, or any modifications or analogs thereof.

BCN and its derivatives are disclosed in WO/2011/136645. DIFO, DIFO2 and DIFO 3 are disclosed in US 2009/0068738. DIBO is disclosed in WO 2009/067663. DIBO may optionally be sulfated (S-DIBO) as disclosed in J. Am. Chem. Soc. 2012, 134, 5381. BARAC is disclosed in J. Am. Chem. Soc. 2010, 132, 3688-3690 and US 2011/0207147. ADIBO derivatives are disclosed in WO/2014/189370.

The stretcher unit may thus comprise an optionally substituted triazole group formed by a reaction between a (cyclic) alkyne group and an azide group of a GalNAz group incorporated at a terminal non-reducing GlcNAc of the targeting unit.

### VI) Specificity Units

The term “specificity unit” or Sₚ may refer to any group, moiety or linker portion capable of linking R₇ or L₁ (if present) to L₂ (if present), to R₈ (if present) or to the targeting unit.

The specificity unit may, in some embodiments, be cleavable. Thereby it can confer cleavability to the linker unit.

The specificity unit may comprise a labile bond configured to be cleavable in suitable conditions. It may thus confer specificity to the cleavability of the conjugate. For example, the stretcher unit may be cleavable only after the cleavage of the specificity unit.

The specificity unit can be, for example, a monopeptide, dipeptide, tripeptide, tetrapeptide, pentapeptide, hexapeptide, heptapeptide, octapeptide, nonapeptide, decapeptide, undecapeptide or dodecapeptide unit. Each Sₚ unit independently may have the formula XIXa or XIXb denoted below in the square brackets:

[chemical structure]

wherein R¹⁹ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, (CH₂)₄NHCOCH₃, —(CH₂)₄NHCHO, —(CH₂)₃NHCONH₂, —(CH₂)₄NHCONH₂, —CH₂CH₂CH(OH) CH₂NH₂, 2-pyridylmethyl-, 3-pyridylmethyl-, 4-pyridylmethyl-, phenyl, cyclohexyl,

[chemical structure]

In some embodiments, the specificity unit can be enzymatically cleavable by one or more enzymes, including a cancer or tumor-associated protease, to liberate the Galectin inhibitor.

In certain embodiments, the specificity unit can comprise natural amino acids. In other embodiments, the specificity unit can comprise non-natural amino acids. Illustrative specificity units are represented by formulas (XX)-(XXII):

[chemical structure]

wherein R²⁰ and R²¹ are as follows:

| R²⁰ | R²¹ |
|---|---|
| Benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| [chemical structure] | (CH₂)₃NHCONH₂; |
| benzyl | methyl; |
| benzyl | (CH₂)₃NHC(═NH)NH₂; |

[chemical structure]

Formula XXI

wherein R²⁰, R²¹ and R²² are as follows:

| R²⁰ | R²¹ | R²² |
|---|---|---|
| benzyl | benzyl | (CH₂)₄NH₂; |
| isopropyl | benzyl | (CH₂)₄NH₂; and |
| H | benzyl | (CH₂)₄NH₂ |

[chemical structure]

Formula XXII

wherein R²⁰, R²¹, R²² and R²³ are as follows:

| R²⁰ | R²¹ | R²² | R²³ |
|---|---|---|---|
| H | benzyl | isobutyl | H; and |
| methyl | isobutyl | methyl | isobutyl |

Exemplary specificity units include, but are not limited to, units of formula XX wherein R²⁰ is benzyl and R²¹ is —(CH₂)₄NH₂; R²⁰ is isopropyl and R₂₁ is —(CH₂)₄NH₂; or R²⁰ is isopropyl and R₂₁ is —(CH₂)₃NHCONH₂. Another exemplary specificity unit is a specificity unit of formula XXI wherein R²⁰ is benzyl, R²¹ is benzyl, and R²² is —(CH₂)₄NH₂.

Useful specificity units can be designed and optimized in their selectivity for enzymatic cleavage by a particular enzyme, for example, a tumour-associated protease. In one embodiment, the specificity unit is cleavable by cathepsin B, C and D, or a plasmin protease.

In an embodiment, the specificity unit is a dipeptide, tripeptide, tetrapeptide or pentapeptide. When R¹⁹, R²⁰, R²¹, R²² or R²³ is other than hydrogen, the carbon atom to which R¹⁹, R²⁰, R²¹, R²² or R²³ is attached is chiral. Each carbon atom to which R¹⁹, R²⁰, R²¹, R²² or R²³ is attached may be independently in the (S) or (R) configuration.

In an embodiment, the specificity unit comprises or is valine-citrulline (vc or val-cit). In another embodiment, the the specificity unit unit is phenylalanine-lysine (i.e. fk). In yet another embodiment, the specificity unit comprises or is N-methylvaline-citrulline. In yet another embodiment, the specificity unit comprises or is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and isonepecotic acid.

### VII) Spacer Units

The term “spacer unit” may refer to any group, moiety or linker portion capable of linking R₇ to Sₚ (if present), L₂ (if present) or the targeting unit. Various types of spacer units may be suitable, and many are known in the art.

Spacer units may be of two general types: non self-immolative or self-immolative. A non self-immolative spacer unit is one in which part or all of the spacer unit remains bound to the Galectin inhibitor moiety after cleavage, for example enzymatic cleavage, of a specificity unit from the conjugate. Examples of a non self-immolative spacer unit include, but are not limited to a (glycine-glycine) spacer unit and a glycine spacer unit. When a conjugate containing a glycine-glycine spacer unit or a glycine spacer unit undergoes enzymatic cleavage via an enzyme (e.g., a tumour-cell associated-protease, a cancer-cell-associated protease or a lymphocyte-associated protease), a glycine-glycine-R₇— Galectin inhibitor moiety or a glycine-R₇-Galectin inhibitor moiety is cleaved from —Sₚ-L2-R₈-T (whichever, if any, of Sₚ-L2-R₈ is present). In one embodiment, an independent hydrolysis reaction takes place within the target cell, cleaving the glycine-R₇-Galectin inhibitor moiety bond and liberating the Galectin inhibitor (and R₇).

In some embodiments, the non self-immolative spacer unit (-L₁-) is -Gly-. In some embodiments, the non self-immolative spacer unit (-L₁-) is -Gly-Gly-.

However, the spacer unit may also be absent.

Alternatively, a conjugate containing a self-immolative spacer unit can release -D, i.e. the Galectin inhibitor, or D-R₇—. In the context of this specification, the term “self-immolative spacer unit” may refer to a bifunctional chemical moiety that is capable of covalently linking together two spaced chemical moieties into a stable tripartite molecule. It may spontaneously separate from the second chemical moiety if its bond to the first moiety is cleaved.

In some embodiments, the spacer unit is a p-aminobenzyl alcohol (PAB) unit (see Schemes 1 and 2 below) the phenylene portion of which is substituted with Qₘ wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

[chemical structure]

[chemical structure]

In some embodiments, the spacer unit is a PAB group that is linked to -Sₚ-, -L₂-, —R₈- or -T via the amino nitrogen atom of the PAB group, and connected directly to —R₇— or to -D via a carbonate, carbamate or ether group. Without being bound by any particular theory or mechanism, Scheme 1 depicts a possible mechanism of release of a PAB group which is attached directly to -D or R⁷ via a carbamate or carbonate group.

In Scheme 1, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

Without being bound by any particular theory or mechanism, Scheme 2 depicts a possible mechanism of Galectin inhibitor release of a PAB group which is attached directly to -D or to —R₇-D via an ether or amine linkage, wherein D may include the oxygen or nitrogen group that is part of the Galectin inhibitor.

In Scheme 2, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

Other examples of self-immolative spacer units include, but are not limited to, aromatic compounds that are electronically similar to the PAB group such as 2-aminoimidazol-5-methanol derivatives and ortho or para-aminobenzylacetals. Other possible spacer units may be those that undergo cyclization upon amide bond hydrolysis, such as substituted and unsubstituted 4-aminobutyric acid amides, appropriately substituted bicyclo[2.2.1] and bicyclo[2.2.2] ring systems and 2-aminophenylpropionic acid amides. Elimination of amine-containing Galectin inhibitors that are substituted at the α-position of glycine are also examples of self-immolative spacers.

In an embodiment, the spacer unit is a branched bis(hydroxymethyl)-styrene (BHMS) unit as depicted in Scheme 3, which can be used to incorporate and release multiple Galectin inhibitors.

[chemical structure]

In Scheme 3, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and n is 0 or 1. The alkyl, alkenyl and alkynyl groups, whether alone or as part of another group, can be optionally substituted.

In some embodiments, the -D moieties are the same. In yet another embodiment, the -D moieties are different.

In an embodiment, the spacer unit is represented by any one of Formulas (XXIII)-(XXV):

[chemical structure]

wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted;

[chemical structure]

### VIII) Further Linker Units

The linker unit may, in some embodiments, comprise a polymer moiety. Such polymer moieties are described e.g. in WO 2015/189478.

In an embodiment, the linker unit L comprises a moiety represented by the formula XXVI, or L is represented by the formula XXVI:

—Y—(CH₂)ₒ—O]_{q}-P— Formula XXVI

wherein

P is a polymer selected from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan, or a derivative thereof;

o is in the range of 1 to 10;

q is at least 1; and

each Y is independently selected from the group consisting of S, NH and 1,2,3-triazolyl, wherein 1,2,3-triazolyl is optionally substituted.

In the above formula, P may be linked to T and Y to D, i.e. the Galectin inhibitor. Y may be linked to D directly, or further groups, moieties or units may be present between Y and D.

Dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan each comprise at least one hydroxyl group. The presence of the at least one hydroxyl group allows the linking of one or more substituents to the polymer as described herein. Many of these polymers also comprise saccharide units that may be further modified, e.g. oxidatively cleaved, to introduce functional groups to the polymer. P may thus also be a polymer derivative.

In this specification, the term “saccharide unit” should be understood as referring to a single monosaccharide moiety.

In this specification, the term “saccharide” should be understood as referring to a monosaccharide, disaccharide or an oligosaccharide.

The value of q may depend e.g. on the polymer, on the Galectin inhibitor, the linker unit, and the method of preparing the conjugate. Typically, a large value of q may led to higher efficiency of the conjugate; on the other hand, a large value of q may in some cases affect other properties of the conjugate, such as pharmacokinetic properties or solubility, adversely. In an embodiment, q is in the range of 1 to about 300, or in the range of about 10 to about 200, or in the range of about 20 to about 100, or in the range of about 20 to about 150. In an embodiment, q is in the range of 1 to about 20, or in the range of 1 to about 15 or in the range of 1 to about 10. In an embodiment, q is 1, 2, 3, 4, 5, 6, 7, 8, 9, 10, 11, 12, 13, 14, 15, 16, 17, 18, 19, or 20. In an embodiment, q is 2-16. In an embodiment, q is in the range of 2 to 10. In other embodiments, q is in the range of 2 to 6; 2 to 5; 2 to 4; 2 or 3; or 3 or 4.

In an embodiment, about 25-45% of carbons of the polymer bearing a hydroxyl group are substituted by a substituent of the formula D-Y—(CH₂)ₙ—O—.

In embodiments in which the polymer comprises a plurality of saccharide units, the ratio of q to the number of saccharide units of the polymer may be e.g. 1:20 to 1:3 or 1:4 to 1:2.

In an embodiment, o is 1, 2, 3, 4, 5, 6, 7, 8, 9 or 10. In an embodiment, o is in the range of 2 to 9, or in the range of 3 to 8, or in the range of 4 to 7, or in the range of 1 to 6, or in the range of 2 to 5, or in the range of 1 to 4.

Each o may, in principle, be independently selected. Each o in a single conjugate may also be the same.

In an embodiment, Y is S.

In an embodiment, Y is NH.

In an embodiment, Y is 1,2,3-triazolyl. In this specification, the term “1,2,3-triazolyl” should be understood as referring to 1,2,3-triazolyl, or to 1,2,3-triazolyl which is substituted. In an embodiment, the 1,2,3-triazolyl is a group formed by click conjugation comprising a triazole moiety. Click conjugation should be understood as referring to a reaction between an azide and an alkyne yielding a covalent product—1,5-disubstituted 1,2,3-triazole—such as copper(I)-catalysed azide-alkyne cycloaddition reaction (CuAAC). Click conjugation may also refer to copper-free click chemistry, such as a reaction between an azide and a cyclic alkyne group such as dibenzocyclooctyl (DBCO). “1,2,3-triazolyl” may thus also refer to a group formed by a reaction between an azide and a cyclic alkyne group, such as DBCO, wherein the group comprises a 1,2,3-triazole moiety.

In an embodiment, the linker unit L comprises a moiety represented by the formula XXVII, or L is represented by the formula XXVII

—Y′—(CH₂)ₚ—S—(CH₂)ₒ—O]_{q}—P— Formula XXVII

wherein

P is a polymer selected from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan, or a derivative thereof;

q is at least 1;

o is in the range of 1 to 10;

p is in the range of 1 to 10; and

each Y′ is independently selected from the group consisting of NH and 1,2,3-triazolyl, wherein 1,2,3-triazolyl is optionally substituted.

In the context of Formula XXVII, each of P, o and q may be as defined for Formula XXVI.

In an embodiment, p is 3, 4, 5, 6, 7, 8, 9 or 10. In an embodiment, p is in the range of 3 to 4, or in the range of 3 to 5, or in the range of 3 to 6, or in the range of 3 to 7, or in the range of 3 to 8, or in the range of 3 to 9. Each p may, in principle, be independently selected. Each p in a single conjugate may also be the same.

In an embodiment, Y′ is selected from the group consisting of NH and 1,2,3-triazolyl.

In an embodiment, P is a polymer derivative comprising at least one saccharide unit.

In an embodiment, P is a polymer derivative comprising at least one saccharide unit, and the polymer derivative is bound to the targeting unit (for example, an antibody) via a bond formed by a reaction between at least one aldehyde group formed by oxidative cleavage of a saccharide unit of the polymer derivative and an amino group of the targeting unit.

In an embodiment, the saccharide unit is a D-glucosyl, D-mannosyl, D-galactosyl, L-fucosyl, D-N-acetylglucosaminyl, D-N-acetylgalactosaminyl, D-glucuronidyl, or D-galacturonidyl unit, or a sulphated derivative thereof.

In an embodiment, the D-glucosyl is D-glucopyranosyl.

In an embodiment, the polymer is selected from the group consisting of dextran, mannan, pullulan, hyaluronic acid, hydroxyethyl starch, chondroitin sulphate, heparin, heparin sulphate, amylose, amylopectin, chitosan, cyclodextrin, pectin and carrageenan. These polymers have the added utility that they may be oxidatively cleaved so that aldehyde groups are formed.

In an embodiment, the polymer is dextran.

In this specification, “dextran” should be understood as referring to a branched glucan composed of chains of varying lengths, wherein the straight chain consists of a α-1,6 glycosidic linkages between D-glucosyl (D-glucopyranosyl) units. Branches are bound via α-1,3 glycosidic linkages and, to a lesser extent, via α-1,2 and/or α-1,4 glycosidic linkages. A portion of a straight chain of a dextran molecule is depicted in the schematic representation below.

[chemical structure]

“D-glucosyl unit” should be understood as referring to a single D-glucosyl molecule. Dextran thus comprises a plurality of D-glucosyl units. In dextran, each D-glucosyl unit is bound to at least one other D-glucosyl unit via a α-1,6 glycosidic linkage, via a α-1,3 glycosidic linkage or via both.

Each D-glucosyl unit of dextran comprises 6 carbon atoms, which are numbered 1 to 6 in the schematic representation below. The schematic representation shows a single D-glucosyl unit bound to two other D-glucosyl units (not shown) via α-1,6 glycosidic linkages.

[chemical structure]

Carbons 2, 3 and 4 may be substituted by free hydroxyl groups. In D-glucosyl units bound to a second D-glucosyl unit via a α-1,3 glycosidic linkage, wherein carbon 3 of the D-glucosyl unit is bound via an ether bond to carbon 1 of the second D-glucosyl unit, carbons 2 and 4 may be substituted by free hydroxyl groups. In D-glucosyl units bound to a second D-glucosyl unit via a α-1,2 or α-1,4 glycosidic linkage, wherein carbon 2 or 4 of the D-glucosyl unit is bound via an ether bond to carbon 1 of the second D-glucosyl unit, carbons 3 and 4 or 2 and 3, respectively, may be substituted by free hydroxyl groups.

A skilled person will understand that other polymers described in this specification also contain free hydroxyl groups bound to one or more carbon atoms and have also other similar chemical properties.

Carbohydrate nomenclature is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 293).

In this specification, the term “Ficoll” refers to an uncharged, highly branched polymer formed by the co-polymerisation of sucrose and epichlorohydrin.

In an embodiment, the polymer is a dextran derivative comprising at least one D-glucosyl unit;

o is in the range of 3 to 10;

Y is S;

the dextran derivative comprises at least one aldehyde group formed by oxidative cleavage of a D-glucosyl unit; and the dextran derivative is bound to the targeting unit (for example, an antibody) via a bond formed by a reaction between at least one aldehyde group of the dextran and an amino group of the targeting unit.

Saccharide units of the polymer, for instance the D-glucosyl units of dextran, may be cleaved by oxidative cleavage of a bond between two adjacent carbons substituted by a hydroxyl group. The oxidative cleavage cleaves vicinal diols, such as D-glucosyl and other saccharide units in which two (free) hydroxyl groups occupy vicinal positions. Saccharide units in which carbons 2, 3 and 4 are substituted by free hydroxyl groups may thus be oxidatively cleaved between carbons 2 and 3 or carbons 3 and 4. Thus a bond selected from the bond between carbons 2 and 3 and the bond between carbons 3 and 4 may be oxidatively cleaved. D-glucosyl units and other saccharide units of dextran and other polymers may be cleaved by oxidative cleavage using an oxidizing agent such as sodium periodate, periodic acid and lead(IV) acetate, or any other oxidizing agent capable of oxidatively cleaving vicinal diols.

Oxidative cleavage of a saccharide unit forms two aldehyde groups, one aldehyde group at each end of the chain formed by the oxidative cleavage. In the conjugate, the aldehyde groups may in principle be free aldehyde groups. However, the presence of free aldehyde groups in the conjugate is typically undesirable. Therefore the free aldehyde groups may be capped or reacted with an amino group of the targeting unit, or e.g. with a tracking molecule.

In an embodiment, the polymer derivative is bound to the targeting unit via a bond formed by a reaction between at least one aldehyde group formed by oxidative cleavage of a saccharide unit of the polymer derivative and an amino group of the targeting unit.

In an embodiment, the polymer derivative may also be bound to the targeting unit via a group formed by a reaction between at least one aldehyde group formed by oxidative cleavage of a saccharide unit of the polymer derivative and an amino group of the targeting unit.

The aldehyde group formed by oxidative cleavage readily reacts with an amino group in solution, such as an aqueous solution. The resulting group or bond formed may, however, vary and is not always easily predicted and/or characterised. The reaction between at least one aldehyde group formed by oxidative cleavage of a saccharide unit of the polymer derivative and an amino group of the targeting unit may result e.g. in the formation of a Schiff base. Thus the group via which the polymer derivative is bound to the targeting unit may be e.g. a Schiff base (imine) or a reduced Schiff base (secondary amine).

### IX) Conjugates

In exemplary embodiments, the conjugate is represented by Formula C:

[D-R₇-L₁-Sₚ-L₂-R₈-]ₙ-T Formula C

wherein D, R₇, L₁, Sₚ, L₂, R₈, n and T are selected from the embodiments described in Table 1.

TABLE 1
Conjugate units and embodiments.

| Unit | Embodiments |
|---|---|
| D, Galectin | a. 33DFTG, |
| inhibitor | b. 6′-acyl-33DFTG, |
| | c. 6 ′-succinyl-33DFTG, |
| | d. di-6-acyl-33DFTG, or |
| | e. di-6-succinyl-33DFTG |
| R₇, a group | a. —C (═O) NH—, |
| covalently | b. —C (═O) O—, |
| bonded to the | c. —NHC (═O)—, |
| Galectin | d. —OC (═O)—, |
| inhibitor | e. —OC (═O) O—, |
| | f. —NHC (═O) O—, |
| | g. —OC (═O) NH—, |
| | h. —NHC (═O) NH, |
| | i. —NH—, |
| | j. —O—, |
| | k. —S—, |
| | l. 1,2,3-triazolyl, or |
| | m. absent |
| L₁, a spacer | a. a C₁₋₁₂ alkyl, |
| unit | b. a substituted C₁₋₁₂ alkyl, |
| | c. a C₅₋₂₀ aryl, |
| | d. a substituted C₅₋₂₀ aryl, |
| | e. a PEG₁₋₅₀ polyethylene glycol |
| | moiety, |
| | f. a substituted PEG₁₋₅₀ polyethylene |
| | glycol moiety, |
| | g. a branched PEG₂₋₅₀ polyethylene |
| | glycol moiety, |
| | h. a substituted branched PEG₂₋₅₀ |
| | polyethylene glycol moiety, |
| | i. a PAB group, or |
| | j. absent |
| Sₚ, a | a. dipeptide, |
| specificity | b. tripeptide, |
| unit | c. tetrapeptide, |
| | d. valine-citrulline, |
| | e. phenylalanine-lysine, |
| | f. valine-alanine, |
| | g. valine-serine, |
| | h. a hydrazone, |
| | i. an ester, |
| | j. a disulfide, |
| | k. a glycoside, or |
| | l. absent |
| L₂, a | a. a C₁₋₁₂ alkyl, |
| stretcher | b. a substituted C₁₋₁₂ alkyl, |
| unit | c. a C₅₋₂₀ aryl, |
| covalently | d. a substituted C₅₋₂₀ aryl, |
| bonded to the | e. a PEG₁₋₈₀ polyethylene glycol |
| targeting | moiety, |
| unit | f. a substituted PEG₁₋₈₀ polyethylene |
| | glycol moiety, |
| | g. a branched PEG₂₋₈₀ polyethylene |
| | glycol moiety, |
| | h. a substituted branched PEG₂₋₅₀ |
| | polyethylene glycol moiety, |
| | i. a moiety represented by the |
| | formula XXVI, |
| | j. a moiety represented by the |
| | formula XXVII, or |
| | k. absent |
| R₈, a group | a. —C (═O) NH—, |
| covalently | b. —C (═O) O—, |
| bonded to the | c. —NHC (═O)—, |
| targeting | d. —OC (═O)—, |
| unit | e. —OC (═O) O—, |
| | f. —NHC (═O) O—, |
| | g. —OC (═O) NH—, |
| | h. —NHC (═O) NH, |
| | i. —NH—, |
| | j. —O—, |
| | k. —S—, |
| | l. 1,2,3-triazolyl, or |
| | m. absent |
| n, number | of about 1, 2, 3, 4, 6, 8, 10, 12, 14, 16, |
| D-L moieties | 18, 20, 22, 24, 28, 30, 32, 36, 40, 44, |
| per targeting | 48, 56, 64, 72, 80, 90, or 100 |
| unit | |
| T, targeting | a. antibody, or |
| unit | b. peptide |

In some embodiments, the conjugate is according to Formula C and Table 1 and selected from the group of:
[D(a)-R₇(m)-L₁(j)-Sₚ(i)-L₂(b)-R₈(k)]ₙ(8)-T(a),
[D(a)-R₇(m)-L₁(j)-Sₚ(i)-L₂(b)-R₈(k)]ₙ(16)-T(a),
[D(a)-R₇(m)-L₁(j)-Sₚ(i)-L₂(b)-R₈(l)]ₙ(2)-T(a),
[D(a)-R₇(m)-L₁(j)-Sₚ(i)-L₂(b)-R₈(l)]ₙ(4)-T(a),
[D(b)-R₇(m)-L₁(j)-Sₚ(i)-L₂(b)-R₈(k)]ₙ(8)-T(a),
[D(b)-R₇(m)-L₁(j)-Sₚ(i)-L₂(b)-R₈(k)]ₙ(16)-T(a),
[D(b)-R₇(m)-L₁(j)-Sₚ(i)-L₂(b)-R₈(l)]ₙ(2)-T(a),
[D(b)-R₇(m)-L₁(j)-Sₚ(i)-L₂(b)-R₈(l)]ₙ(4)-T(a), [D(c)-R₇(m)-L₁(j)-Sₚ(i)-L₂(b)-R₈(k)]ₙ(8)-T(a),
[D(c)-R₇(m)-L₁(j)-Sₚ(i)-L₂(b)-R₈(k)]ₙ(16)-T(a),
[D(c)-R₇(m)-L₁(j)-Sₚ(i)-L₂(b)-R₈(l)]ₙ(2)-T(a),
[D(c)-R₇(m)-L₁(j)-Sₚ(i)-L₂(b)-R₈(l)]ₙ (4)-T(a), [D(d)-R₇(a)-L₁(b)-Sₚ(l)-L₂(k)-R₈ (k)]ₙ (8)-T(a),
[D(d)-R₇(m)-L₁(j)-Sₚ(l)-L₂(k)-R₈(k)]ₙ(16)-T(a),
[D(d)-R₇(m)-L₁(j)-Sₚ(l)-L₂(k)-R₈(l)]ₙ(2)-T(a),
[D(d)-R₇(m)-L₁(j)-Sₚ(l)-L₂(k)-R₈(l)]ₙ(4)-T(a),
[D(e)-R₇ (i)-L₁(j)-Sₚ(l)-L₂(b)-R₈(k)]ₙ(8)-T(a),
[D(e)-R₇ (i)-L₁(j)-Sₚ(l)-L₂(b)-R₈(k)]ₙ(16)-T(a),
[D(e)-R₇(i)-L₁(j)-Sₚ(l)-L₂(b)-R₈(l)]ₙ(2)-T(a),
[D(e)-R₇(i)-L₁(j)-Sₚ(l)-L₂(b)-R₈(l)]ₙ(4)-T(a);

wherein the letters in parentheses refer to the embodiments in Table 1.

The conjugate may be selected from the group consisting of conjugates represented by Formulas Ca to Ce:

[chemical structure]

[chemical structure]

[chemical structure]

wherein T is the targeting unit; and

n is at least 1, or about 1, 2, 3, 4, 6, 8, 10, 12, 14, 16, 18, 20, 22, 24, 28, 30, 32, 36, 40, 44, 48, 56, 64, 72, 80, 90, or 100.

The conjugate may be any conjugate described in this specification; a skilled person may derive various conjugates by combining any one of the above units and Galectin inhibitors described in this specification.

### X) Compositions and Methods

A pharmaceutical composition comprising the conjugate according to one or more embodiments described in this specification is disclosed.

The pharmaceutical composition may further comprise one or more further components, for example a pharmaceutically acceptable carrier. Examples of suitable pharmaceutically acceptable carriers are well known in the art and may include e.g. phosphate buffered saline solutions, water, oil/water emulsions, wetting agents. and liposomes. Compositions comprising such carriers may be formulated by methods well known in the art. The pharmaceutical composition may further comprise other components such as vehicles, additives, preservatives, other pharmaceutical compositions administrated concurrently, and the like.

In an embodiment, the pharmaceutical composition comprises an effective amount of the conjugate according to one or more embodiments described in this specification.

In an embodiment, the pharmaceutical composition comprises a therapeutically effective amount of the conjugate according to one or more embodiments described in this specification.

The term “therapeutically effective amount” or “effective amount” of the conjugate may be understood as referring to the dosage regimen for achieving a therapeutic effect, for example modulating the growth of cancer cells and/or treating a patient's disease. The therapeutically effective amount may be selected in accordance with a variety of factors, including the age, weight, sex, diet and medical condition of the patient, the severity of the disease, and pharmacological considerations, such as the activity, efficacy, pharmacokinetic and toxicology profiles of the particular conjugate used. The therapeutically effective amount can also be determined by reference to standard medical texts, such as the Physicians Desk Reference 2004. The patient may be male or female, and may be an infant, child or adult.

The term “treatment” or “treat” is used in the conventional sense and means attending to, caring for and nursing a patient with the aim of combating, reducing, attenuating or alleviating an illness or health abnormality and improving the living conditions impaired by this illness, such as, for example, with a cancer disease.

In an embodiment, the pharmaceutical composition comprises a composition for e.g. oral, parenteral, transdermal, intraluminal, intraarterial, intrathecal, intra-tumoral (i.t.), and/or intranasal administration or for direct injection into tissue. Administration of the pharmaceutical composition may be effected in different ways, e.g. by intravenous, intraperitoneal, subcutaneous, intramuscular, intra-tumoral, topical or intradermal administration.

A conjugate according to one or more embodiments described in this specification or a pharmaceutical composition comprising the conjugate according to one or more embodiments described in this specification for use as a medicament is disclosed.

A conjugate according to one or more embodiments described in this specification or a pharmaceutical composition comprising the conjugate according to one or more embodiments described in this specification for use in decreasing immunosuppressive activity in a tumour is disclosed.

A conjugate according to one or more embodiments described in this specification or a pharmaceutical composition comprising the conjugate according to one or more embodiments described in this specification for use in the treatment, modulation and/or prophylaxis of the growth of tumour cells in a human or animal is also disclosed.

A conjugate according to one or more embodiments described in this specification or a pharmaceutical composition comprising the conjugate according to one or more embodiments described in this specification for use in the treatment of cancer is disclosed.

The cancer may be selected from the group of leukemia, lymphoma, breast cancer, prostate cancer, ovarian cancer, colorectal cancer, gastric cancer, squamous cancer, small-cell lung cancer, head-and-neck cancer, multidrug resistant cancer, glioma, melanoma, and testicular cancer. However, other cancers and cancer types may also be contemplated.

In an embodiment, the conjugate is a conjugate for use in the inhibition of inflammation, inhibition of fibrosis, inhibition of angiogenesis, inhibition of infection, inhibition of HIV-1 infection, or inhibition of autoimmune disease or autoimmune reactions in the target tissue.

In an embodiment, the conjugate is a conjugate for use in the inhibition of any Galectin-mediated condition in the target tissue.

The conjugate may be administered in combination with a cancer immunotherapeutic agent.

In principle, the cancer immunotherapeutic agent may be any cancer immunotherapeutic agent.

In an embodiment, the cancer immunotherapeutic agent is an immune receptor-targeting antibody, an immune checkpoint inhibitor, an anti-immune checkpoint molecule, anti-PD-1, anti-PD-L1 antibody, anti-CTLA-4 antibody, a cancer-targeting molecule, or a targeting unit capable of binding an immune checkpoint molecule.

In an embodiment, the cancer immunotherapeutic agent is an immune receptor-targeting antibody, an immune checkpoint inhibitor, an anti-immune checkpoint molecule, anti-PD-1, anti-PD-L1 antibody, anti-CTLA-4 antibody, or a targeting unit capable of binding an immune checkpoint molecule.

A method of treating, modulating and/or prophylaxis of the growth of tumour cells in a human or animal is also disclosed.

A method of treating, modulating, prophylaxis and/or inhibiting inflammation, fibrosis, angiogenesis, infection, HIV-1 infection, or autoimmune disease or autoimmune reactions in a target tissue in a human or animal is also disclosed.

A method of inhibiting any Galectin-mediated condition in a target tissue in a human or animal is also disclosed.

The method may comprise administering the conjugate according to one or more embodiments described in this specification or the pharmaceutical composition according to one or more embodiments described in this specification to a human or animal in an effective amount.

The tumour cells may be selected from the group of leukemia cells, lymphoma cells, breast cancer cells, prostate cancer cells, ovarian cancer cells, colorectal cancer cells, gastric cancer cells, squamous cancer cells, small-cell lung cancer cells, head-and-neck cancer cells, multidrug resistant cancer cells, and testicular cancer cells.

In an embodiment, the conjugate is administered in combination with a cancer immunotherapeutic agent.

A method for preparing the conjugate according to one or more embodiments described in this specification is disclosed. The method may comprise conjugating the Galectin inhibitor to the targeting unit.

In the context of the method, the Galectin inhibitor may be any Galectin inhibitor described in this specification, for example a Galectin inhibitor represented by any one of formulas II-IX.

In an embodiment of the method, the conjugate is represented by formula I, and the method comprises conjugating the Galectin inhibitor to the linker unit; and conjugating the targeting unit to the linker unit, thus forming a conjugate represented by formula I.

In an embodiment of the method, the conjugate is represented by formula X, and the method comprises conjugating the Galectin inhibitor to the spacer unit; conjugating the targeting unit to the stretcher unit; and conjugating the spacer unit and the stretcher unit to each other, optionally via a specificity unit, thus forming a conjugate represented by formula X.

In the context of the method, the targeting unit, the linker unit, the spacer unit, the stretcher unit, and or the specificity unit may be according to any one of the embodiments described in this specification, for example in any one of the sections II)-VIII).

Anything disclosed above in the context of the conjugate may also be understood as being disclosed in the context of the method(s).

The activity of the conjugates may be measured by their inhibition of Galectin function and/or interaction by numerous methods known in the art.

The ability of the Galectin inhibitor(s) to enter cells of the target tissue may be measured by various functional assays, for example by employing flow cytometry.

Inhibition of immune suppression may be measured by for example in vitro assays using target cells and immune cells, and measuring cell kill activity, cellular activation, cytokine production, or the like. Examples of suitable immune cell assay methods are well known for a person skilled in the art.

### EXAMPLES

### Example 1. Conjugation of Linker to 33DFTG

[chemical structure]

Scheme E1-1: 5.3 mg (8 μmol) of 3,3′-dideoxy-3,3′-bis-[4-(3-fluorophenyl)-1H-1,2,3-triazol-1-yl]-1,1′-sulfanediyl-di-β-D-galactopyranoside (33DFTG, or TD139; MedChemExpress Europe, Sollentuna, Sweden), 2.8 molar excess of succinic anhydride in pyridine (21 μl) and 79 μl pyridine were stirred at room temperature (RT) for 3.5 hours. The crude reaction mixture was analysed by MALDI-TOF mass spectrometry (MALDI-TOF MS) with Bruker Ultraflex III TOF/TOF instrument (Bruker Daltonics, Bremen, Germany) using 2,5-dihydroxybenzoic acid (DHB) matrix, showing expected masses for 6-succinyl-33DFTG (FIG. 1, m/z 771 [M+Na]⁺) and di-6-succinyl-DFTG (FIG. 1, m/z 871 [M+Na]⁺). The reaction was quenched by adding 0.5 ml ethanol.

The products were purified by Äkta purifier (GE Healthcare) HPLC instrument with Sdex peptide SE column (10×300 mm, 13 μm (GE Healthcare)) in aqueous ammonium acetate buffer. di-6-succinyl-DFTG was recovered in one of the collected fractions and detected by MALDI-TOF MS similarly as above (FIG. 2).

[chemical structure]

Scheme E1-2: 2 μmol di-6-succinyl-33DFTG, 3 molar excess of DBCO-amine, 5 molar excess of HBTU, 2 μl DIPEA and 100 μl DMF were stirred at RT for overnight. The DBCO-di-6-succinyl-33DFTG products were purified by Äkta purifier (GE Healthcare) HPLC instrument with Gemini 5 μm NX-C18 reverse phase column (4.6×250 mm, 110 Å (Phenomenex)) eluted with acetonitrile gradient in aqueous ammonium acetate. The fractions were analysed by MALDI-TOF MS similarly as above, showing expected masses for mono-DBCO-di-6-succinyl-33DFTG (m/z 1129 [M+Na]⁺) and di-DBCO-di-6-succinyl-33DFTG (FIG. 3, m/z 1387 [M+Na]⁺).

### Example 2. Conjugation of Linker-Modified 33DFTG to Cancer-Targeting Antibody

[chemical structure]

Scheme E2-1: 4 mg of anti-HER2 antibody Trastuzumab (Herceptin, Roche) was first digested with endoglycosidase S2 according to manufacturers instructions (Glycinator; Genovis, Lund, Sweden) and then incubated with 0.4 mg recombinant Y289L mutant bovine β1,4-galactosyltransferase and 1.3 mg UDP-GalNAz (both from Thermo, Eugene, USA) in the presence of Mn²⁺ containing buffer at +37° C. overnight. Azide-to-antibody ratio was determined by Fabricator enzyme digestion according to manufacturer's instructions (Genovis) and MALDI-TOF MS essentially as described (Satomaa et al. 2018. Antibodies 7(2), 15). FIG. 4 shows the heavy chain Fc domains of trastuzumab after endoglycosidase digestion (FIG. 4A; at m/z 24001 for the non-fucosylated glycoform and at m/z 24148 for the fucosylated glycoform) and then after galactosyltransferase reaction (FIG. 4B; at m/z 24249 for the non-fucosylated glycoform and at m/z 24394 for the fucosylated glycoform), with all the peaks arising from successfully azide-labeled antibody fragments, demonstrating that the azide-to-antibody ratio was 2.

[chemical structure]

Scheme E2-2: DAR=2 azido-trastuzumab is incubated with 10 molar excess of mono-DBCO-di-6-succinyl-33DFTG in phosphate-buffered saline (PBS) at RT for 1 hour to react essentially all azide groups with the DBCO-linker compound via a triazole bond. Excess small molecules are removed by repeated filtration through Amicon centrifugal filter tubes with 10 kDa cutoff and addition of PBS. Drug-to-antibody ratio (DAR) is determined by Fabricator enzyme digestion (Genovis, Lund, Sweden) and MALDI-TOF MS essentially as described (Satomaa et al. 2018. Antibodies 7(2), 15). The product is characterized as DAR=2 6-succinyl-33DFTG-trastuzumab by observing that all detectable heavy chain Fc fragments have gained +1107 m/z compared to non-conjugated DAR=2 azido-trastuzumab.

### Example 3. Inhibition of Galection Interaction with Cancer Cells by 33DFTG

SKOV-3 ovarian adenocarcinoma cells (ATCC, Manassas, Va., USA) were cultured according to ATCC's instructions and incubated in the presence of 2 μM 33DFTG for 3 days or DMSO carrier control in parallel. After the incubation, cells were stained with Alexa Fluor 488-conjugated human recombinant Galectin-1, and Alexa Fluor 488-conjugated human recombinant Galectin-3 (both from Abcam, Cambridge, United Kingdom; and both at 10 μg/ml) for 45 minutes at +4° C. Cells were washed and stored on ice in the dark until analysed by FACSAriaII flow cytometer. FIG. 5 and the numerical results tabulated below show that binding of the Galectins to the cells was clearly decreased by the treatment.

| Mean fluorescence | Untreated | Control | 2 μM |
|---|---|---|---|
| intensity | SKOV-3 cells | treatment | 33DFTG |
| Unstained control | 94 | 97 | 97 |
| Galectin-1 | 426 | 439 | 186 |
| Galectin-3 | 1740 | 1940 | 165 |

In another experiment, HSC-2 oral cavity squamous cell carcinoma cells (head-and-neck cancer) were cultured for two days in standard culture conditions, after which 2 μM 33DFTG was added to the cell culture medium and the cells cultured for 2 more days with the inhibitor. In parallel, untreated cells were cultured in normal cell culture medium. For flow cytometry analysis, cells were detached with trypsin, washed, and stained with AlexaFluor488-conjugated Galectin-1 and Galectin-3 proteins as above. FACS was performed as above. FIG. 6 and the numeric results tabulated below show that binding of the Galectins was clearly decreased by the treatment.

| Mean fluorescence | Untreated | Control | 2 μM |
|---|---|---|---|
| intensity | HSC-2 cells | treatment | 33DFTG |
| Unstained control | 89 | 70 | n/a |
| Galectin-1 | 611 | 521 | 226 |
| Galectin-3 | 1529 | 1247 | 129 |

n/a: not analyzed

### Example 4. Inhibition of Galectin Interaction with Target Cells by DAR=2 6-succinyl-33DFTG-trastuzumab

[chemical structure]

SKOV-3 ovarian carcinoma cells are cultured as described above and incubated in the presence of DAR=2 6-succinyl-33DFTG-trastuzumab for 3-4 days. The ADC is internalized to the cells via binding to HER2 receptors on the cell surface and the payload is released inside the cells (Scheme E4). After the incubation, cells are stained with AlexaFluor488-conjugated human recombinant Galectin-1 and Galectin-3, and analyzed by FACS as above. ADC concentration is increased until detectable Galectin inhibition is reached.

### Example 6. Maleimide-Linker Conjugated 33DFTG

[chemical structure]

Scheme E6-1. Di-6-succinyl-33DFTG is combined with 2 molar excess of N-(2-aminoethyl)maleimide (Sigma) and 2 molar excess of HBTU in DMF with 1% DIPEA and stirred at RT overnight. The products are purified by Äkta purifier (GE Healthcare) HPLC instrument with Gemini 5 μm NX-C18 reverse phase column (4.6×250 mm, 110 Å (Phenomenex)) eluted with acetonitrile gradient in aqueous ammonium acetate buffer. The fractions are analysed by MALDI-TOF MS similarly as above, showing expected mass for mono-maleimido-di-6-succinyl-33DFTG at m/z 993 [M+Na]+.

### Example 7. Inhibition of Galectin Interaction in Tumors in Combination with Immune Checkpoint Inhibition in Tumour-Bearing Animals by DAR=2 and DAR=8 33DFTG-Trastuzumab

DAR=2 6-succinyl-33DFTG-trastuzumab is prepared as described above.

[chemical structure]

Scheme E7-1: For preparation of DAR=8 6-succinyl-33DFTG-trastuzumab conjugate, the hinge region disulphides are reduced by TCEP as described (Satomaa et al. 2018) and combined with 8 molar excess of mono-maleimido-di-6-succinyl-33DFTG in PBS at RT for 2 hours, after which unconjugated drug-linker is removed by repeated filtration through Amicon centrifugal filter tubes with 10 kDa cutoff and addition of PBS.

HER2-positive cancer cells are cultured as described above, injected subcutaneously to mice (about 1-10 million cells/mouse in Matrigel), and allowed to form xenograft tumors of about 100 mm³. Mice are divided into groups that receive daily 100 μl intravenous injections of either I) PBS (vehicle control), II) 10 mg/kg trastuzumab in PBS (antibody control), III) 10 mg/kg DAR=2 6-succinyl-33DFTG-trastuzumab in PBS, or IV) 10 mg/kg DAR=8 6-succinyl-33DFTG-trastuzumab in PBS. Lower Galectin activity in groups III-VI compared to groups I-II is observed as sign of successful tumour-targeted inhibition of GlcNAc-transferases in vivo, leading to lower immunosuppression of antibody therapy and greater anti-cancer therapeutic activity. The ADC therapy is further combined with immune checkpoint inhibitor therapy by intravenous injection of therapeutic dose of anti-PD-1 antibody or anti-PD-L1 antibody in further groups of mice for even greater anti-cancer activity.

### Example 8. Preparation of Conjugates

[chemical structure]

[chemical structure]

Schemes E8-1 and E8-2: 33DFTG was coupled with S-acetylmercaptosuccinic anhydride as described above. The product E8-1 had correct m/z of 845.15 [M+Na]⁺ in MALDI-TOF MS. After selective deprotection of the thiol group with aqueous hydroxylamine the product had correct m/z of 803.33 [M+Na]⁺ in MALDI-TOF MS, thus enabling it to be conjugated from the thiol group to a targeting unit.

[chemical structure]

Scheme E8-3. To increase affinity especially to Galectin-9, dimeric galectin inhibitor was prepared from 6-succinyl-33DFTG via a PEG spacer. Amino-PEG5-amine (Cat. No. CP-1115) was obtained from Conju-Probe and amidated to 6-succinyl-33DFTG essentially as described above in DMF in presence of HBTU and DIPEA. The product had correct m/z of 1763.56 [M+Na]⁺ in MALDI-TOF MS. Diamino-PEG5-di-(6-succinyl-33DFTG) was purified from the reaction mixture with RP-HPLC as described above and the purified product had correct m/z of 1763.31 [M+Na]⁺ in MALDI-TOF MS.

Finally, several ADCs were prepared with DBCO-modified 33DFTG-derivative-linkers shown in Schemes E8-4 to E8-6 below, by conjugating them separately to DAR=2 and DAR=4 azide-modified trastuzumab in PBS essentially as described above.

[chemical structure]

[chemical structure]

[chemical structure]

FIG. 7 shows the characterization of the ADCs with Fabricator digestion and MALDI-TOF MS of the isolated antibody fragments, performed essentially as in Satomaa et al. 2018, Antibodies 7(2):15. FIG. 8 shows HIC-HPLC chromatography of trastuzumab-DBCO-PEG4-VC-PAB-DMAE-33DFTG DAR=4 ADC, performed essentially as in Satomaa et al. 2018, which demonstrated that the hydrophobicity/hydrophilicity properties of the ADC were similar as that of the clinically approved ADC Adcetris.

### Example 9. Functional Assays

To study if the ADCs were configured to release the galectin inhibitor payload upon internalization to target cell lysosome, 33DFTG-linker compounds comprising the Val-Cit dipeptide sequence were tested and found to be sensitive to cleavage by recombinant lysosomal protease cathepsin B (R&D Systems, Cat. No. 953-CY-010), liberating the free 33DFTG payload upon the enzyme treatment. First, the enzyme was activated by 1 hour incubation with 5 mM dithiotreitol in 50 mM Na-acetate pH 5. Then, 11 ng of the activated enzyme (nominal activity >27 μmol/min) was incubated with 2 nmol of DBCO-PEG4-VC-PAB-DMAE-33DFTG linker-payload in 50 mM Na-acetate pH 5 for 4 hours at +37° C. and additionally for 3 days at room temperature. The reaction products were analyzed by MALDI-TOF MS in DHB matrix as described above. Before the reaction, the m/z of the linker-payload was detected at 1724.71 [M+Na]⁺. After the reaction, correct m/z 831.31 [M+Na]⁺ of linker fragment was detected, corresponding to cleavage by the lysosomal enzyme (Scheme E9-1).

[chemical structure]

To study if the galectin inhibitor payload had activity in vivo to decrease the immunosuppressive activity of cancer cells and to boost immune responses against cancer, an in vivo trial with cancer cell xenografts was performed. It was demonstrated that the galectin inhibitor has such activity in vivo and that it could be combined with immunotherapy for improved treatment outcome. The trial was performed as follows: repeated dose 1.5 mg/kg pembrolizumab (Keytruda, Merck), repeated dose 1.8 mg/kg 33DFTG and repeated dose of the combination of the above treatments were evaluated against NCI-N87 cancer cell line tumors. The study was performed by Inovotion SAS (La Tronche, France). Fertilized chicken eggs were incubated at 37.5° C. with 50% relative humidity for 9 days (E9), when the chorioallantoic membrane (CAM) was dropped down by drilling a small hole through the eggshell into the air sac, and a 1 cm² window was cut in the eggshell above the CAM. The NCI-N87 cell line was cultivated in RPMI-1640 medium supplemented with 10% FBS and 1% penicillin/streptomycin. On day E9, cells were detached by trypsin, washed with complete medium and suspended in graft medium. An inoculum of 2 million cells was added onto the CAM of each egg. On day 10 (E10), tumors began to be detectable. Living grafted eggs were randomized into groups and were then treated on day E10, E11.5, E13, E14.5 and E17 (five doses) by dropping 100 μl of vehicle (PBS) and compounds (alone or in combination) onto the tumor. On day 18 (E18) the upper portion of the CAM was removed, washed in PBS and then directly transferred in PFA (fixation for 48 h). The tumors were then carefully cut away from normal CAM tissue and weighed. Eggs were checked at each treatment time, or at least every two days, for viability during the study. At the end of the study, the number of dead embryos was counted and combined with the observation of eventual visible macroscopic abnormalities (observation done during the sample collection) to evaluate the toxicity.

The results of the in vivo trial are shown in Table 2 below. There were no major differences in % alive egg embryos, since the level of % alive egg embryos was deemed normal. Compared to both PBS control group and the pembrolizumab treatment group, the combination treatment group had lower mean tumor size (27.7 mg compared to 30.6 mg and 33.5 mg, respectively) and thus higher therapeutic efficacy. However, neither pembrolizumab alone or 33DFTG alone showed significant difference compared to the control group.

TABLE 2
in vivo trial results.

| | Ctrl | Pembroliz- | | 33DFTG + |
|---|---|---|---|---|
| Group | (PBS) | umab | 33DFTG | Pembroliz-umab |
| Mean tumor | 30.6 | 33.5 | 35.8 | 27.7 |
| size (mg) | | | | |
| SEM | 2.3 | 4.5 | 4.5 | 4.6 |
| n | 13 | 8 | 9 | 8 |
| % alive | 81 | 73 | 64 | 67 |

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A product, a method, or a use, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1-18. (canceled)

19. A conjugate comprising
a targeting unit for delivery to a target tissue, and
a Galectin inhibitor for inhibiting Galectin interaction within the target tissue, wherein
the Galectin inhibitor is conjugated to the targeting unit.

20. The conjugate according to claim 19, wherein the conjugate is represented by formula I:
[D-L]ₙ-T Formula I
wherein D is the Galectin inhibitor, T is the targeting unit, L is a linker unit linking D to T at least partially covalently, and n is at least 1.

21. The conjugate according to claim 19, wherein the Galectin inhibitor is selected from the group of galactose, a 3-substituted galactose, a β-D-galactoside, a galactoside, a 3-substituted galactoside, a β-D-galactoside, a 3-substituted β-D-galactoside, lactose, a 3′-substituted lactose, a lactoside, a 3′-substituted lactoside, N-acetyllactosamine, a 3′-substituted N-acetyllactosamine, an N-acetyllactosaminide, a 3′-substituted N-acetyllactosaminide, N,N′-di-N-acetyllactosediamine, a 3′-substituted N,N′-di-N-acetyllactosediamine, an N,N′-di-N-acetyllactosediaminide, a 3′-substituted N,N′-di-N-acetyllactosediaminide, a taloside, a 3′-substituted taloside, a β-D-taloside, a 3′-substituted β-D-taloside, a mannoside, a 3′-substituted mannoside, a β-D-mannoside, a 3′-substituted β-D-mannoside, thiodigalactose (TDG), a 3-substituted thiodigalactose, a 3,3′-disubstituted thiodigalactose, 3,3′-dideoxy-3,3′-bis-[4-(3-fluorophenyl)-1H-1,2,3-triazol-1-yl]-1,1′-sulfanediyl-di-β-D-galactopyranoside (33DFTG or TD139), 6-acyl-33DFTG, 6-succinyl-33DFTG, di-6-acyl-33DFTG, di-6-succinyl-33DFTG, a 6-substituted 33DFTG, a 6,6′-disubstituted 33DFTG, (E)-methyl-2-phenyl-4-(β-D-galactopyranosyl)-but-2-enoate, Galβ1-4Fuc, a 3′-substituted Galβ1-4Fuc, GM-CT-01, GR-MD-02, a pectin, reduced pectin, modified citrus pectin, GCS-100, a poly-N-acetyllactosaminide, lactulose, a lactuloside, a 3′-substituted lactulose, a 3′-substituted lactuloside, lactulosyl-L-leucine, a 3′-substituted lactulosyl-L-leucine, a Galectin-binding peptide, a Galectin-binding peptidomimetic, anginex (βpep-25), 6DBF7, DB16, DB21, PTX008 (0118/OTX008), PTX009 (1097a), a Galectin-binding molecule that inhibits Galectin-Galectin ligand interaction, a Galectin-binding antibody, a Galectin-binding antibody fragment, a Galectin-binding nanobody, an RNAi inhibiting Galectin expression, a soluble Galectin, a soluble Galectin fragment, an oxidized Galectin, an oxidized Galectin fragment, and any analog, modification, combination, or multivalent combination thereof.

22. The conjugate according to claim 19, wherein the Galectin inhibitor is masked with a removable masking substituent, such that the Galectin inhibitor is capable of binding to a Galectin only after removal of the removable masking substituent.

23. The conjugate according to claim 19, wherein the Galectin inhibitor is represented by Formula II: [chemical structure]
wherein:
W is O, S, NH, NY₁, CH₂, CY₁H or C(Y₁)₂;
X is O, S, S(═O), S(═O)₂, NH, NY₁, CH₂, CY₁H, C(Y₁)₂ or a bond;
R₁ is H, a saccharide, a saccharide substituted with L′, Z, M, a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl, a substituted C₆-C₂₀ aryl or L′;
R₂ is H, OH, OZ, OM, NHCOCH₃, NHZ, NHM or L′;
R₃ is H, OH, OZ, OM, NHZ, NHM, L′ or Y₃;
R₄ is H, OH, OZ, OM or L′;
R₅ is H, CH₂, a saccharide, a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl, a substituted C₆-C₂₀ aryl or a bond;
Y₅ is either absent or H, OH, OZ, OM or L′;
L′ is a bond to L;
M is a removable masking substituent, independently selected from the group of an acetal, hemiacetal, ketal, hemiketal, imino, formyl, acyl, carboxy, thiocarboxy, thiolocarboxy, thionocarboxy, imidic acid, hydroxamic acid, ester, acyloxy, oxycarboyloxy, amino, amido, thioamido, acylamido, aminocarbonyloxy, ureido, guanidino, tetrazolyl, imino, amidine, nitro, nitroso, azide, cyano, isocyano, cyanato, isocyanato, thiocyano, isothiocyano, sulfhydryl, thioether, disulfide, sulfine, sulfone, sulfinic acid, sulfonic acid, sulfinate, sulfonate, sulfinyloxy, sulfonyloxy, sulfate, sulfamyl, sulfonamido, sulfamino, sulfonamino, phospho, phosphinic acid, phosphonate, phosphoric acid, phosphate, phosphorous acid, phosphite, phosphoramidite, or phosphoramidate substituent, or a glycoside or peptide substituent;
each Z is independently selected from the group of a C₁-C₁₀ acyl or a substituted C₁-C₁₀ acyl;
each Y₁ is independently selected from a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl and a substituted C₆-C₂₀ aryl;
with the proviso that the Galectin inhibitor D contains not more than one L′; and
wherein
Y₃ is a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl and a substituted C₆-C₂₀ aryl, an azide, or a structure described by any one of formulas FY3-A, FY3-B, FY3-C, FY3-D, FY3-E, or FY3-F: [chemical structure]
wherein the arrow shows the bond to rest of the structure; [chemical structure]
wherein the arrow shows the bond to rest of the structure; and wherein R¹, R², R³, R⁴ and R⁵ are independently selected from the group of H, optionally substituted alkyl groups, halogens, optionally substituted alkoxy groups, OH, substituted carbonyl groups, optionally substituted acyloxy groups, and optionally substituted amino groups; wherein two, three, four or five of R¹, R², R³, R⁴ and R⁵ in adjacent positions may be linked to form one or more rings, and the remaining of R¹, R², R³, R⁴ and R⁵ is/are independently selected from the above group; [chemical structure]
wherein the arrow shows the bond to rest of the structure; and wherein
Y₃a is either O or NH,
Y₃b is selected from the group of CO, SO₂, SO, PO₂, PO, and CH₂, or is a bond, and
Y₃c is selected from the group of:
a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens, or
b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamino group, a phenyl group substituted with at least one hydroxy group, a phenyl group substituted with at least one carbonyl group and a phenyl group substituted with at least one substituted carbonyl group, or
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, or
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group; [chemical structure]
wherein the arrow shows the bond to rest of the structure; and Y_{3d} is selected from the group of CH₂, CO, SO₂, and phenyl or is a bond; Ria is selected from the group of D-galactose, C3-substituted D-galactose, C3-1,2,3-triazol-1-yl-substituted D-galactose, H, a C₁-C₁₀ alkyl, a C₁-C₁₀ alkenyl, a C₆-C₂₀ aryl, an imino group and a substituted imino group; Y3e is selected from the group of an amino group, a substituted amino group, an alkyl group, a substituted alkyl group, an alkoxy group, a substituted alkoxy group, an alkylamino group, a substituted alkylamino group, a substituted naphthyl group, a thienyl group, and a substituted thienyl group: wherein said substituent is one or more selected from the group consisting of halogen, alkoxy, alkyl, nitro, sulfo, amino, hydroxy or carbonyl group; [chemical structure]
wherein the arrow shows the bond to rest of the structure, and wherein
Y_{3f} is either CONH or a 1H-1,2,3-triazole ring; and
Y_{3g} is selected from the group of an alkyl group of at least 4 carbons, an alkenyl group of at least 4 carbons, an alkynyl group of at least 4 carbons, a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, a carbamoyl group substituted with an substituted aryl group, a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group, a phenyl group substituted with at least one trifluoromethoxy group, a phenyl group substituted with at least one sulfo group, a phenyl group substituted with at least one hydroxy group, a phenyl group substituted with at least one carbonyl group, a phenyl group substituted with at least one substituted carbonyl group, a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, a naphthyl group substituted with at least one substituted carbonyl group, a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, a heteroaryl group substituted with at least one substituted carbonyl group, a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group; [chemical structure]
wherein the arrow shows the bond to rest of the structure; and wherein
Y₃ₕ is NH, CH₂, NRₓ or a bond; Y₃ᵢ is CO, SO, SO₂, PO or PO₂H; Y₃ⱼ is selected from the group of an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, an alkyl group of at least 4 carbon atoms Substituted with a halogen, a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxyl group, a phenyl group substituted with a carbonyl group, a phenyl group substituted with a substituted carbonyl group and a phenyl amino group; Rib is H, a saccharide, an alkyl group, an alkenyl group, or an aryl group and wherein Rx is H, an alkyl group, an alkenyl group, an aryl group, a heteroaryl group or a heterocycle.

24. The conjugate according to claim 19, wherein the Galectin inhibitor is represented by formula III: [chemical structure]
wherein:
W′ and W″ are each independently selected from the group of O, S, N, NH, NY₁, CH, CH₂, CY₁H and C(Y₁)₂;
R₂′ is H, OH, OZ, OM, NHCOCH₃, NHZ, NHM or L′;
R₃′ is H, OH, OZ, OM, NHCOCH₃, NHZ, NHM, L′ or Y₃′;
R₄′ is either absent or H, OH, OZ, OM and L′;
R₅′ and R₆′ are each independently either absent or selected from the group of H, CH₂, a saccharide, a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl, a substituted C₆-C₂₀ aryl and a bond;
Y₅′ and Y₆′ are each independently either absent or selected from the group of H, OH, OZ, OM and L′;
Y₃′ is a C₁-C₁₀alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl and a substituted C₆-C₂₀ aryl, an azide, or a structure described by any one of formulas FY3-A, FY3-B, FY3-C, FY3-D, FY3-E or FY3-F as described in claim 23;
and wherein the other substituents are as described in claim 23;
with the proviso that the Galectin inhibitor contains not more than one L′.

25. The conjugate according to claim 19, wherein the Galectin inhibitor is represented by any one of Formulas IV to IX: [chemical structure]
wherein R¹, R², R³, R⁴ and R⁵ are independently selected from the group of H, optionally substituted alkyl groups, halogens, optionally substituted alkoxy groups, OH, substituted carbonyl groups, optionally substituted acyloxy groups, and optionally substituted amino groups; wherein two, three, four or five of R¹, R², R³, R⁴ and R⁵ in adjacent positions may be linked to form one or more rings, and the remaining of R¹, R², R³, R⁴ and R⁵ is/are independently selected from the above group; [chemical structure]
wherein:
Y₃a and Y₃a′ are independently either O or NH,
Y₃b and Y₃b′ are independently selected from the group of CO, SO₂, SO, PO₂, PO, and CH₂, or is a bond, and
Y₃c and Y₃c′ are independently selected from the group of:
a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; or
b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamino group, a phenyl group substituted with at least one hydroxy group, a phenyl group substituted with at least one carbonyl group and a phenyl group substituted with at least one substituted carbonyl group, or
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, or
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group; [chemical structure]
wherein Y_{3d} is selected from the group of CH₂, CO, SO₂, and phenyl or is a bond; Ria is selected from the group of D-galactose, C3-substituted D-galactose, C3-1,2,3-triazol-1-yl-substituted D-galactose, H, a C₁-C₁₀ alkyl, a C₁-C₁₀ alkenyl, a C₆-C₂₀ aryl, an imino group and a substituted imino group; Y₃ₑ is selected from the group of an amino group, a substituted amino group, an alkyl group, a substituted alkyl group, an alkoxy group, a substituted alkoxy group, an alkylamino group, a substituted alkylamino group, a substituted naphthyl group, a thienyl group, and a substituted thienyl group: wherein said substituent is one or more selected from the group consisting of halogen, alkoxy, alkyl, nitro, sulfo, amino, hydroxy or carbonyl group; [chemical structure]
wherein Y_{3f} and Y_{3f}′ are each independently either CONH or a 1H-1,2,3-triazole ring; Y_{3g} and Y_{3g}′ are each independently selected from the group of an alkyl group of at least 4 carbons, an alkenyl group of at least 4 carbons, an alkynyl group of at least 4 carbons, a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, a carbamoyl group substituted with an substituted aryl group, a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group, a phenyl group substituted with at least one trifluoromethoxy group, a phenyl group substituted with at least one sulfo group, a phenyl group substituted with at least one hydroxy group, a phenyl group substituted with at least one carbonyl group, a phenyl group substituted with at least one substituted carbonyl group, a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, a naphthyl group substituted with at least one substituted carbonyl group, a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, a heteroaryl group substituted with at least one substituted carbonyl group, a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group; [chemical structure]
wherein Y₃ₕ is NH, CH₂, NRₓ or a bond; Y₃ᵢ is CO, SO, SO₂, PO or PO₂H; Y₃ⱼ is selected from the group of an alkyl group of at least 4 carbon atoms, an alkenyl group of at least 4 carbon atoms, an alkyl or alkenyl group of at least 4 carbon atoms Substituted with a carboxy group, an alkyl group of at least 4 carbon atoms substituted with both a carboxy group and an amino group, an alkyl group of at least 4 carbon atoms Substituted with a halogen, a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxyl group, a phenyl group substituted with a carbonyl group, a phenyl group substituted with a substituted carbonyl group and a phenyl amino group; Rib is H, a saccharide, an alkyl group, an alkenyl group, or an aryl group; and wherein Rx is H, an alkyl group, an alkenyl group, an aryl group, a heteroaryl group or a heterocycle;
and wherein:
Y₅ is either absent or H, OH, OZ, OM or L′;
X is O, S, S(═O), S(═O)₂, NH, NY₁, CH₂, CY₁H, C(Y₁)₂ or a bond;
Y₅′ is absent or selected from the group of H, OH, OZ, OM and L′;
each Z is independently selected from the group of a C₁-C₁₀ acyl or a substituted C₁-C₁₀ acyl;
M is a removable masking substituent, independently selected from the group of an acetal, hemiacetal, ketal, hemiketal, imino, formyl, acyl, carboxy, thiocarboxy, thiolocarboxy, thionocarboxy, imidic acid, hydroxamic acid, ester, acyloxy, oxycarboyloxy, amino, amido, thioamido, acylamido, aminocarbonyloxy, ureido, guanidino, tetrazolyl, imino, amidine, nitro, nitroso, azide, cyano, isocyano, cyanato, isocyanato, thiocyano, isothiocyano, sulfhydryl, thioether, disulfide, sulfine, sulfone, sulfinic acid, sulfonic acid, sulfinate, sulfonate, sulfinyloxy, sulfonyloxy, sulfate, sulfamyl, sulfonamido, sulfamino, sulfonamino, phospho, phosphinic acid, phosphonate, phosphoric acid, phosphate, phosphorous acid, phosphite, phosphoramidite, or phosphoramidate substituent, or a glycoside or peptide substituent;
L′ is a bond to L; and,
each Y₁ is independently selected from a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl and a substituted C₆-C₂₀ aryl.

26. The conjugate according to claim 19, wherein the linker unit is configured to release the Galectin inhibitor into an extracellular space of the target tissue after the conjugate is delivered and/or bound to the target tissue.

27. The conjugate according to claim 19, wherein the targeting unit is an antibody, such as a tumour-targeting, a cancer-targeting antibody and/or an immune cell-targeting antibody; a peptide; an aptamer; or a glycan.

28. The conjugate according to claim 19, wherein:
the targeting unit is a cancer-targeting antibody selected from the group of bevacizumab, tositumomab, etanercept, trastuzumab, adalimumab, alemtuzumab, gemtuzumab ozogamicin, efalizumab, rituximab, infliximab, abciximab, basiliximab, palivizumab, omalizumab, daclizumab, cetuximab, panitumumab, epratuzumab, 2G12, lintuzumab, nimotuzumab and ibritumomab tiuxetan, or the antibody is selected from the group of an anti-EGFR1 antibody, an epidermal growth factor receptor 2 (HER2/neu) antibody, an anti-CD22 antibody, an anti-CD30 antibody, an anti-CD33 antibody, an anti-Lewis y antibody, an anti-CD20 antibody, an anti-CD3 antibody, an anti-PSMA antibody, an anti-TROP2 antibody, an anti-AXL antibody; or
the targeting unit comprises or is an immune receptor-targeting antibody selected from the group of nivolumab, pembrolizumab, ipilimumab, atezolizumab, avelumab, durvalumab, BMS-986016, LAG525, MBG453, OMP-31M32, JNJ-61610588, enoblituzumab (MGA271), MGD009, 8H9, MEDI9447, M7824, metelimumab, fresolimumab, IMC-TR1 (LY3022859), lerdelimumab (CAT-152), LY2382770, lirilumab, IPH4102, 9B12, MOXR 0916, PF-04518600 (PF-8600), MED6383, MEDI0562, MEDI6469, INCAGN01949, GSK3174998, TRX-518, BMS-986156, AMG 228, MED11873, MK-4166, INCAGN01876, GWN323, JTX-2011, GSK3359609, MEDI-570, utomilumab (PF-05082566), urelumab, ARGX-110, BMS-936561 (MDX-1203), varlilumab, CP-870893, APX005M, ADC-1013, lucatumumab, Chi Lob 7/4, dacetuzumab, SEA-CD40, R07009789, MEDI9197; or
the targeting unit comprises or is a molecule selected from the group of an immune checkpoint inhibitor, an anti-immune checkpoint molecule, anti-PD-1, anti-PD-L1 antibody, anti-CTLA-4 antibody, a cancer-targeting molecule, or a targeting unit capable of binding an immune checkpoint molecule, the immune checkpoint molecule being selected from the group of: lymphocyte activation gene-3 (LAG-3, CD223), T cell immunoglobulin-3 (TIM-3), poly-N-acetyllactosamine, T (Thomsen-Friedenreich antigen), Globo H, Lewis c (type 1 N-acetyllactosamine), Galectin-1, Galectin-2, Galectin-3, Galectin-4, Galectin-5, Galectin-6, Galectin-7, Galectin-8, Galectin-9, Galectin-10, Galectin-11, Galectin-12, Galectin-13, Galectin-14, Galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (Υ_{c}), CD25 (IL-2Rα), and arginase.

29. The conjugate according to claim 20, wherein n is in the range of 1 to about 20, or 1 to about 15, or 1 to about 10, or 2 to 10, or 2 to 6, or 2 to 5, or 2 to 4, or 3 to about 20, or 3 to about 15, or 3 to about 10, or 3 to about 9, or 3 to about 8, or 3 to about 7, or 3 to about 6, or 3 to 5, or 3 to 4, or 4 to about 20, or 4 to about 15, or 4 to about 10, or 4 to about 9, or 4 to about 8, or 4 to about 7, or 4 to about 6, or 4 to 5; or about 7-9; or about 8, or 1, 2, 3, 4, 5, 6, 7, 8, 9, 10, 11, 12, 13, 14, 15, 16, 17, 18, 19, or 20; or in the range of 1 to about 1000, or 1 to about 400, or 1 to about 200, or 1 to about 100; or 100 to about 1000, or 200 to about 1000, or 400 to about 1000, or 600 to about 1000, or 800 to about 1000; 100 to about 800, or 200 to about 600, or 300 to about 500; or 20 to about 200, or 30 to about 150, or 40 to about 120, or 60 to about 100; over 8, over 16, over 20, over 40, over 60, over 80, over 100, over 120, over 150, over 200, over 300, over 400, over 500, over 600, over 800, or over 1000; or n is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 52, 54, 56, 58, 60, 62, 63, 64, 66, 68, 70, 72, 74, 76, 78, 80, 82, 84, 86, 88, 90, 92, 94, 96, 98, 100, 110, 120, 130, 140, 150, 160, 170, 180, 190, 200, 220, 240, 260, 280, 300, 350, 400, 450, 500, 550, 600, 650, 700, 750, 800, 850, 900, 950, 1000, 1100, 1200, 1300, 1400, 1500, 1600, 1700, 1800, 1900, 2000, or greater than 2000.

30. The conjugate according to claim 20, wherein L is represented by Formula X:
—R₇-L₁-Sₚ-L₂-R₈— Formula X
wherein:
R₇ is a group covalently bonded to the Galectin inhibitor;
L₁ is spacer unit or absent;
Sₚ is a specificity unit or absent; and
L2 is a stretcher unit covalently bonded to the targeting unit or absent; and
R₈ is absent or a group covalently bonded to the targeting unit.

31. The conjugate according to claim 30, wherein R₇ is selected from the group consisting of:
—C(═O)NH—,
—C(═O)O—,
—NHC(═O)—,
—OC(═O)—,
—OC(═O)O—,
—NHC(═O)O—,
—OC(═O)NH—,
—NHC(═O)NH,
—O—,
—NH—,
1,2,3-triazole, and
—S—; and
R₈ is either absent or selected from:
—C(═O)NH—,
—C(═O)O—,
—NHC(═O)—,
—OC(═O)—,
—OC(═O)O—,
NHC(═O)O—,
—OC(═O)NH—,
—NHC(═O)NH,
—O—,
—NH—,
1,2,3-triazole, and
—S—.

32. A pharmaceutical composition comprising the conjugate according to claim 19.

33. The pharmaceutical composition according to claim 32 for use as a medicament, for use in the modulation or prophylaxis of the growth of tumour cells, for use in the inhibition of any Galectin-mediated condition in the target tissue, or for use in the treatment of cancer.

34. The pharmaceutical composition according to claim 33, wherein the cancer is selected from the group of leukemia, lymphoma, breast cancer, prostate cancer, ovarian cancer, colorectal cancer, gastric cancer, squamous cancer, small-cell lung cancer, head-and-neck cancer, multidrug resistant cancer, glioma, melanoma, and testicular cancer.

35. The pharmaceutical composition for use according to claim 33, wherein the conjugate is administered in combination with a cancer immunotherapeutic agent.

36. A method for preparing the conjugate according to claim 19, the method comprising conjugating the Galectin inhibitor to the targeting unit.
